(19) United States
(12) Patent Application Publication
(10) Pub. No.: US 2017/0158697 A1
(43) Pub. Date: Jun. 8, 2017

(54) Protein Kinase Inhibitors

(71) Applicant: Pharmascience Inc., Montreal, Québec (CA)

(72) Inventors: Alain Laurent, Montreal, Québec (CA); Yannick Rose, Montreal, Québec (CA)

(73) Assignee: Pharmascience Inc., Montreal, Québec (CA)

(21) Appl. No.: 15/039,127

(22) PCT Filed: Nov. 26, 2014
(86) PCT No.: PCT/CA2014/000848

§ 371 Date: May 25, 2016

(30) Foreign Application Priority Data
Nov. 26, 2013 (CA) 2,834,528

(51) Int. Cl. C07D 487/04; A61K 31/5377; A61K 45/06; A61K 31/519

(52) U.S. Cl. CPC C07D 487/04; A61K 31/519; A61K 31/5377; A61K 45/06

(57) ABSTRACT

The present invention relates to a novel family of protein kinase inhibitors, more specifically the present invention is directed to inhibitors of the members of the Tec or Src protein kinase families. The present invention also relates to processes for the preparation of these compounds, to the pharmaceutical composition comprising them, and to their use in the treatment of proliferative, inflammatory, infectious or autoimmune diseases, disorder or condition in which protein kinase activity is implicated.

### FIELD OF INVENTION

The present invention relates to a novel family of protein kinase inhibitors, to the processes for preparation of these compounds, to pharmaceutical compositions comprising them, and to their use in the treatment of proliferative, inflammatory, autoimmune, or infectious diseases, disorders, or conditions associated with kinase function.

### BACKGROUND OF THE INVENTION

Protein kinases are a large group of intracellular and transmembrane signaling proteins in eukaryotic cells (Manning G. et al, (2002) Science, 298: 1912-1934). These enzymes are responsible for transfer of the terminal (gamma) phosphate from ATP to specific amino acid residues of target proteins. Phosphorylation of specific amino acid residues in target proteins can modulate their activity leading to profound changes in cellular signaling and metabolism. Protein kinases can be found in the cell membrane, cytosol and organelles such as the nucleus and are responsible for mediating multiple cellular functions including metabolism, cellular growth and differentiation, cellular signaling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: Tec, Src, Abl, Jak, Csk, Fak, Syk, Fer, Ack and the receptor tyrosine kinase subfamilies including EGFR, FGFR, VEGFR, RET and Eph.

Kinases exert control on key biological processes related to health and disease. Furthermore, aberrant activation or excessive expression of various protein kinases are implicated in the mechanism of multiple diseases and disorders characterized by benign and malignant proliferation, as well as diseases resulting from inappropriate activation of the immune system (Kyttaris V. C., Drug Des. Devel. Ther. 2012, 6:245-50 and Fabbro D. et al. Methods Mol. Biol., 2012, 795:1-34). Thus, inhibitors of select kinases or kinase families are expected to be useful in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, dermatomyositis, pemphigus, and the like.

Tec kinases are a family of non-receptor tyrosine kinases predominantly, but not exclusively, expressed in cells of hematopoietic origin (Bradshaw J. M. Cell Signal., 2010, 22:1175-84). The Tec family includes Tec, Bruton's tyrosine kinase (Btk), inducible T-cell kinase (Itk), resting lymphocyte kinase (Rlk/Txk), and bone marrow-expressed kinase (Bmx/Etk). Btk is important in B-cell receptor signaling and regulation of B-cell development and activation (W. N. Khan et al. Immunity, 1995, 3:283-299 and Satterthwaite A. B. et al. Immunol. Rev. 2000, 175: 120-127). Mutation of the gene encoding BTK in humans leads to X-linked agammaglobulinemia which is characterized by reduced immune function, including impaired maturation of B cells, decreased levels of immunoglobulin and peripheral B cells, diminished T-cell independent immune response (Rosen F. S. et al., N. Engl. J. Med., 1995, 333:431-440; and Lindvall J. M. et al. Immunol. Rev. 2005, 203:200-215). Btk is activated by Src-family kinases and phosphorylates PLC gamma leading to effects on B-cell function and survival. Additionally, Btk is important in signal transduction in response to immune complex recognition by macrophage, mast cells and neutrophils. Btk inhibition is also important in survival of lymphoma cells (Herman SEM. Blood, 2011, 117:6287-6289) suggesting that inhibition of Btk may be useful in the treatment of lymphomas. As such, inhibitors of Btk and related kinases are of great interest as anti-inflammatory as well as anti-cancer agents. Btk is also important for platelet function and thrombus formation suggesting that Btk-selective inhibitors may prove to be useful antithrombotic agents (Liu J. Blood, 2006, 108:2596-603).

Bmx, another Tec family member which has roles in inflammation, cardiovascular disease, and cancer (Cenni B. et al. Int Rev. Immunol. 2012, 31: 166-173) is also important for self-renewal and tumerogenic potential of glioblastoma stem cells (Guryanova O. A. et al. Cancer Cell Cancer Cell 2011, 19:498-511). As such, Bmx inhibitors are expected to be useful in the treatment of various diseases including cancer, cardiovascular disease and inflammation.

The SRC family of tyrosine kinases includes cSRC, Lyn, Fyn, Lck, Hck, Fgr, Blk, Syk, Yrk and Yes. cSRC is critically involved in signaling pathways involved in cancer and is often over-expressed in human malignancies (Kim L. C. et al. (2009) Nat. Rev. Clin. Oncol. 6:587-9). cSRC is involved in signaling downstream of growth factor receptor tyrosine kinases and regulates cell cycle progression suggesting that cSRC inhibition would impact cancer cell proliferation. Furthermore, Src inhibitors or downregulation of Hck sensitize tumor cells to immunotoxins (Lui X. F., Mol. Cancer Ther. 2013, Oct. 21).

Inhibition of SRC family members may be useful in treatments designed to modulate immune function. SRC family members, including Lck, regulate T-cell receptor signal transduction which leads to gene regulation events resulting in cytokine release, survival and proliferation. Thus, inhibitors of Lck may be useful immunosuppressive agents with potential application in graft rejection and T-cell mediated autoimmune disease (Martin et al. Expert Opin. Ther. Pat. 2010, 20:1573-93). The Src family member HCK is implicated in regulation of cytokine production suggesting that inhibition of this kinase may be useful in treatment of inflammatory disease (Smolinska M. J. et al. J. Immunol. 2011; 187:6043-51). Additionally, the Src family kinase Fgr is critical for activation of mast cells and IgE-mediated anaphylaxis suggesting that this kinase is a potential therapeutic target for allergic diseases (Lee J. H. et al. J. Immunol. 2011; 187:1807-15)

Inhibition of kinases using small molecule inhibitors has successfully led to several approved therapeutic agents used in the treatment of a variety of diseases disorders and conditions. Herein, we disclose a novel family of kinase inhibitors. Further, we demonstrate that modifications in compound substitution can influence kinase selectivity and therefore the biological function of that agent.

### SUMMARY OF THE INVENTION

The present invention relates to a novel family of kinase inhibitors. Compounds of this class have been found to have inhibitory activity against members of the Tec or Scr protein kinase families.

One aspect of the present invention is directed to a compound of Formula I:

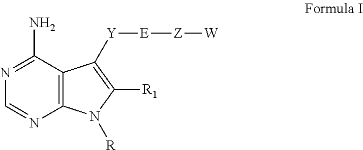

or pharmaceutically acceptable salts, solvates, solvates of salts, stereoisomers, tautomers, isotopes, prodrugs, complexes or biologically active metabolites thereof, wherein

R is selected from the group consisting of:
1) hydrogen,
2) alkyl,
3) heteroalkyl,
4) carbocyclyl,
5) heterocyclyl,
6) aryl, or
7) heteroaryl,

wherein the alkyl, heteroalkyl, carbocyclyl, heterocyclyl, aryl, or heteroaryl are optionally substituted;

R¹ is selected from the group consisting of:
1) hydrogen,
2) alkyl,
3) heteroalkyl,
4) carbocyclyl,
5) heterocyclyl, or
6) halogen,

wherein the alkyl, heteroalkyl, carbocyclyl, or heterocyclyl are optionally substituted;

Y is

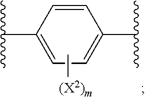

E is oxygen;

Z is

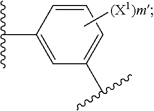

W is
1) —OCH₂R², or
2) —CH₂OR², wherein

R² is substituted or unsubstituted aryl, substituted or unsubstituted heteroaryl;

wherein Y-E-Z—W is

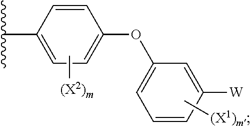

X¹ and X² are independently hydrogen or halogen;

m is an integer from 0 to 4,

m′ is an integer from 0 to 4.

Other embodiments of the present invention include compounds of Formula I, wherein W is selected from the group consisting of:

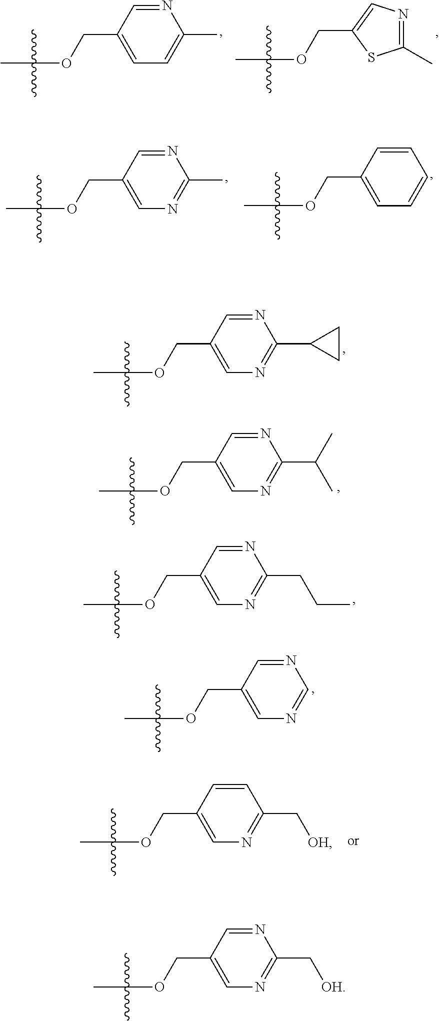

Another embodiment includes compounds of Formula I, wherein Z is selected from the group consisting of:

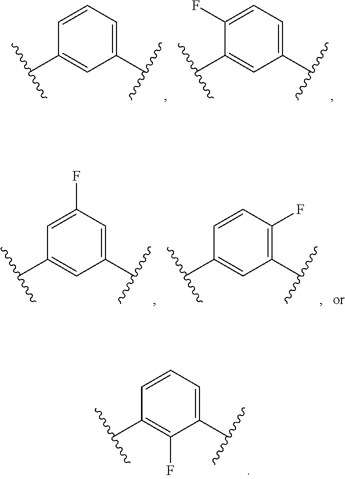 [SMILES: *c1cccc(*)c1 ; *c1ccc(F)c(*)c1 ; *c1cc(*)cc(F)c1 ; *c1ccc(F)c(*)c1 ; *c1cccc(*)c1F]

Another embodiment of the present invention includes compounds of Formula I, wherein Y is

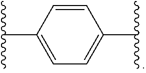 [SMILES: *c1ccc(*)cc1]

Preferred embodiment includes compounds of Formula I, wherein R¹ is hydrogen.

Another embodiment of the present invention includes compounds of Formula I, wherein R is selected from the group consisting of:

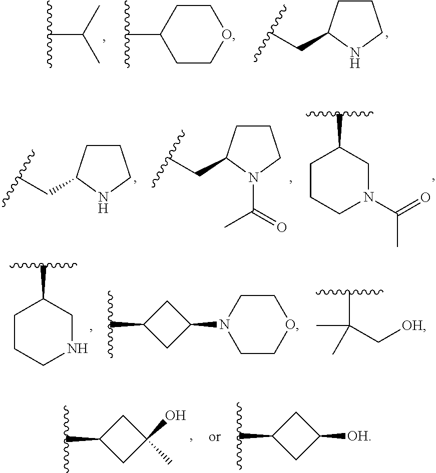

Another embodiment of the present invention includes compounds of Formula II:

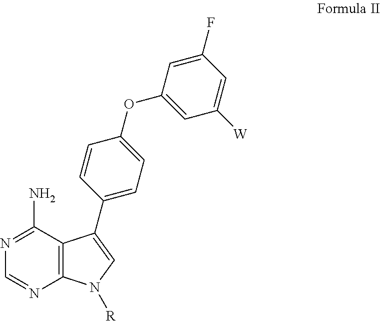

or pharmaceutically acceptable salts, solvates, solvates of salts, stereoisomers, tautomers, isotopes, prodrugs, complexes or biologically active metabolites thereof, wherein

R is selected from the group consisting of:
1) hydrogen,
2) alkyl,
3) heteroalkyl,
4) carbocyclyl,
5) heterocyclyl,
6) aryl, or
7) heteroaryl,

wherein the alkyl, heteroalkyl, carbocyclyl, heterocyclyl, aryl, or heteroaryl are optionally substituted;

W is
1) —OCH₂R², or
2) —CH₂OR², wherein

R² is substituted or unsubstituted aryl, substituted or unsubstituted heteroaryl.

Another embodiment of the present invention includes compounds of Formula II, wherein W is selected from the group consisting of:

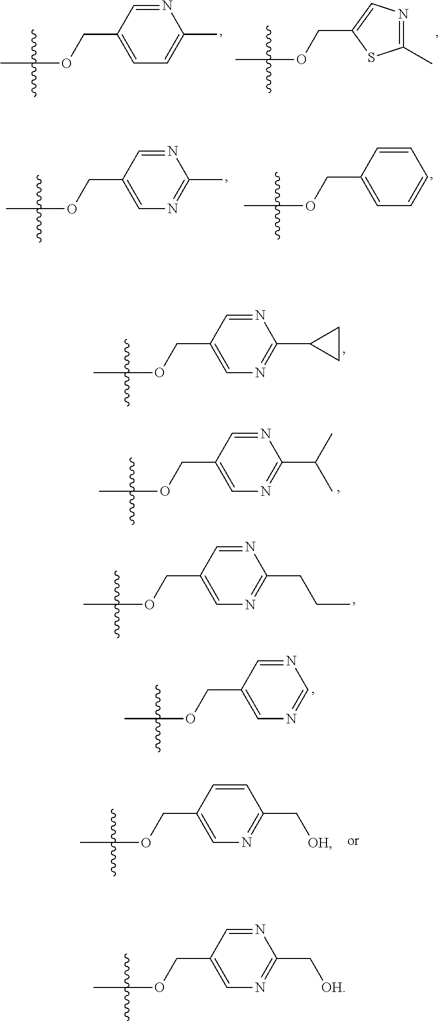

Another embodiment of the present invention includes compounds of Formula II, wherein R is selected from the group consisting of:

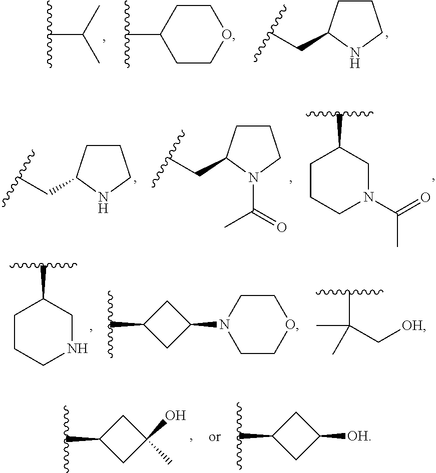

Another aspect of the present invention provides intermediates and their synthesis related to a process of production of compounds of the invention as defined herein, or a pharmaceutically acceptable salt, or solvate, solvates of salts, stereoisomers, tautomers, isotopes, prodrugs, complexes or biologically active metabolites thereof, or a pharmaceutical composition as defined herein.

In another aspect, the present invention relates to a process for preparing a compound of Formula I or Formula II, wherein the process comprises:

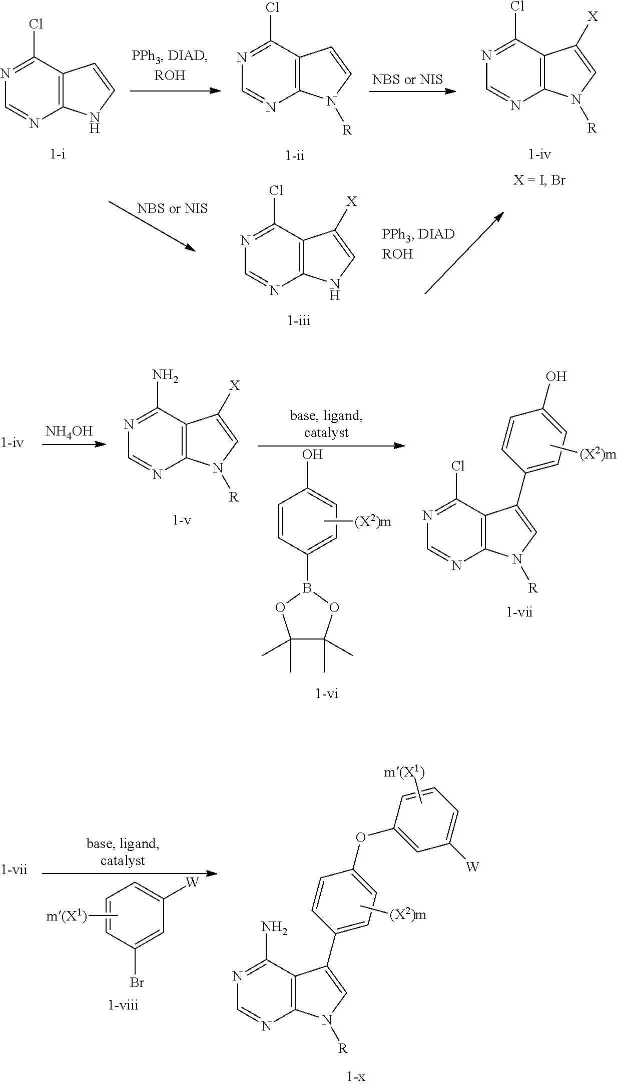

Another aspect of the present invention provides the process for preparing a compound of Formula I or Formula II, wherein the process comprises:

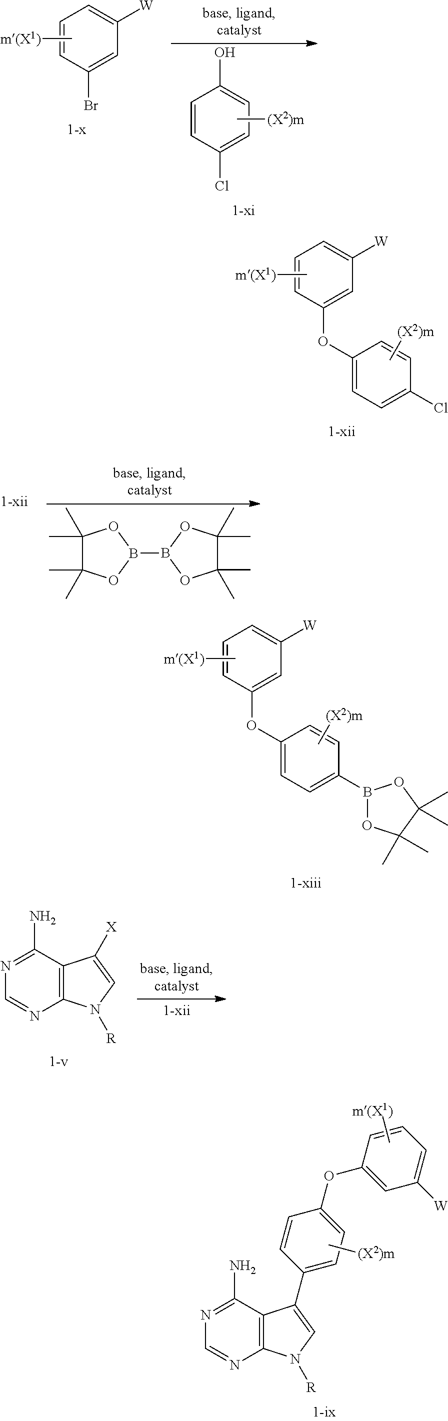

Another aspect of the present invention provides a pharmaceutical composition comprising a compound of Formula I, or Formula II, or a pharmaceutically acceptable salts, solvates, solvates of salts, stereoisomers, tautomers, isotopes, prodrugs, complexes or biologically active metabolites thereof, and at least one pharmaceutically acceptable carrier, diluents, or excipient.

In another aspect, the present invention relates to a compound of the invention as defined herein, or a pharmaceutically acceptable salt, solvates, solvates of salts, stereoisomers, tautomers, isotopes, prodrugs, complexes or biologically active metabolites thereof, or a pharmaceutical composition as defined herein, for use in therapy.

In another aspect, the present invention relates to a compound of the invention as defined herein, or a pharmaceutically acceptable salt or solvate thereof, or a pharmaceutical composition as defined herein, for use in the treatment of subjects suffering from a protein kinase mediated diseases or conditions.

Another aspect of the present invention provides a use of the compound of Formula I, or Formula II, or a pharmaceutically acceptable salt, solvates, solvates of salts, stereoisomers, tautomers, isotopes, prodrugs, complexes or biologically active metabolites thereof, as an inhibitor of protein kinase, more particularly, as an inhibitor of members of the Tec family of kinases.

A further aspect of the present invention provides a use of the compound of Formula I, or Formula II, or a pharmaceutically acceptable salt, solvates, solvates of salts, stereoisomers, tautomers, isotopes, prodrugs, complexes or biologically active metabolites thereof, as an inhibitor of protein kinase, more particularly, as an inhibitor of members of the Src family of kinases.

Another aspect of the present invention provides a use of the compound of Formula I, or Formula II, as an inhibitor of protein kinase, more particularly, as an inhibitor wherein the diseas is a protein kinase mediated disease, disorder, or condition in which Btk kinase activity is implicated.

In another aspect, the present invention relates to the use of a compound of the invention as defined herein, or a pharmaceutically acceptable salt or solvate thereof, in the manufacture of a medicament for use in the treatment of subjects suffering from a protein kinase mediated diseases or conditions.

A further aspect of the present invention provides a pharmaceutically acceptable salt, or solvate thereof, for use in manufacturing of a pharmaceutical composition, for use in treatment of proliferative, inflammatory, infectious, or autoimmune diseases.

Another aspect of the present invention provides a compound, or pharmaceutically acceptable salts, or solvates thereof, or a pharmaceutical composition, as defined in present invention, for use in the treatment of a proliferative disorder, inflammatory, or autoimmune disease. In a particular embodiment, the proliferative disorder, inflammatory, or autoimmune disease is cancer. More particular, is a human cancer.

A further aspect of the present invention provides the use of a compound, or a pharmaceutically acceptable salt or solvate thereof, in the manufacture of a medicament for use in the treatment of a proliferative disorder, such as cancer.

Another aspect of the present invention provides a compound of Formula I, or Formula II, or a pharmaceutically acceptable salts, solvates, solvates of salts, stereoisomers, tautomers, isotopes, prodrugs, complexes, or biologically active metabolites thereof, for use in the treatment of a proliferative, inflammatory, or autoimmune diseases, or disorder state in combination with an agent selected from: an estrogen receptor modulator; an androgen receptor modulator; a retinoid receptor modulator; a cytotoxic agent; an anti-proliferative agent comprises adriamycin, dexamethasone, vincristine, cyclophosphamide, fluorouracil, topotecan, taxol, interferons, or platinum derivatives; an anti-inflammatory agent comprises corticosteroids, TNF blockers, IL-1 RA, azathioprine, cyclophosphamide, or sulfasalazine; a prenyl-protein transferase inhibitor; an HMG-CoA reductase inhibitor; an HIV protease inhibitor; a reverse transcriptase inhibitor; an angiogenesis inhibitor comprises sorafenib, sunitinib, pazopanib or everolimus; an immunomodulatory or immunosuppressive agents comprises cyclosporin, tacrolimus, rapamycin, mycophenolate mofetil, interferons, corticosteroids, cyclophophamide, azathioprine, or sulfasalazine; a PPAR-γ agonist comprising thiazolidinediones; a PPAR-δ agonist; an inhibitor of inherent multidrug resistance; an agent for the treatment of anemia, comprising erythropoiesis-stimulating agents, vitamins or iron supplements; an anti-emetic agent including 5-HT3 receptor antagonists, dopamine antagonists, NK1 receptor antagonist, H1 histamine receptor antagonists, cannabinoids, benzodiazepines, anticholinergic agents or steroids; an agent for the treatment of neutropenia; an immunologic-enhancing agents; a proteasome inhibitors; an HDAC inhibitors; an inhibitor of the chemotrypsin-like activity in the proteasome; a E3 ligase inhibitors; a modulator of the immune system including interferon-alpha, Bacillus Calmette-Guerin (BCG), or ionizing radition (UVB) that can induce the release of cytokines, interleukins, TNF, or induce release of death receptor ligands including TRAIL; a modulator of death receptors TRAIL or TRAIL agonists including humanized antibodies HGS-ETR1 or HGS-ETR2; neurotrophic factors selected from cetylcholinesterase inhibitors, MAO inhibitors, interferons, anti-convulsants, ion channel blockers, or riluzole; Anti-Parkinsonian agents comprising anticholinergic agents or dopaminergic agents, including dopaminergic precursors, monoamine oxidase B inhibitors, COMT inhibitors, dopamine receptor agonists; agents for treating cardiovascular disease comprises beta-blockers, ACE inhibitors, diuretics, nitrates, calcium channel blockers, or statins; agents for treating liver disease comprises corticosteroids, cholestyramine, or interferons; anti-viral agents, including nucleoside reverse transcriptase inhibitors, non-nucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry inhibitors; agents for treating blood disorders comprising corticosteroids, anti-leukemic agents, or growth factors; agents for treating immunodeficiency disorders comprising gamma globulin, adalimumab, etarnecept or infliximab; a HMG-CoA reductase inhibitors including torvastatin, fluvastatin, lovastatin, pravastatin, rosuvastatin, simvastatin, or pitavastatin, or in combination, or sequentially with radiation, or with at list one chemotherapeutic agent.

More preferably the medicament is for the treatment of a proliferative disorder or disease state in combination with a death receptor agonist.

Another aspect of the present invention provides a compound, or pharmaceutically acceptable salts, or solvates thereof, or a pharmaceutical composition as defined in present invention, for use in the treatment of diseases or disorders selected from: cancer, myeloproliferative disorders, lung fibrosis, hepatic fibrosis, cardiovascular diseases: cardiac hypertrophy, cardiomyopathy, restenosis; thrombosis, heart attacks or stroke; alopecia, emphysema; atherosclerosis, psoriasis or dermatological disorders, lupus, multiple sclerosis, macular degeneration, asthma, reactive synoviotides, viral disorders; CNS disorders; auto-immune disorders: glomerulonephritis or rheumatoid arthritis; hormone-related diseases, metabolic disorders; inflammatory diseases; infectious or fungal diseases, malaria or parasitic disorders.

Another aspect of the present invention provides a compound, or pharmaceutically acceptable salts, or solvates thereof, or a pharmaceutical composition, as defined in present invention, for use in the manufacture of a medicament for the treatment of: arthritis, tenosynovial giant cell tumour, pigmented villonodular synovitis, and other reactive synoviotides, bone metastases formation and progression, acute myeloid leukemia, or human cancer, or select subsets of cancer, for example breast tumours and gastric cancer by inhibition of kinase activity.

In another aspect, the present invention relates to a method of treating a disease or condition associated with protein kinase activity, said method comprising administering to a subject a therapeutically effective amount of a compound of the invention as defined herein, or a pharmaceutically acceptable salt or solvate thereof, or a pharmaceutical composition as defined herein.

In another aspect, the present invention provides a method of treating a proliferative disorder, said method comprising administering to a subject a therapeutically effective amount of a compound, or a pharmaceutically acceptable salt or solvate thereof, or a pharmaceutical composition as defined herein. In a particular embodiment, the proliferative disorder is a cancer.

Another aspect of the present invention provides a method of modulating kinase function, the method comprising contacting a cell with a compound of the present invention in an amount sufficient to modulate the enzymatic activity of a given kinase, or kinases from Tec family kinases, thereby modulating the kinase function.

A further aspect of the present invention provides a method of modulating kinase function, the method comprising contacting a cell with a compound of the present invention in an amount sufficient to modulate the enzymatic activity of a given kinase, or kinases from Src family, thereby modulating the kinase function.

Another aspect of the present invention provides a method of inhibiting cell proliferation or survival in vitro or in vivo, said method comprising contacting a cell with an effective amount of a compound as defined herein, or a pharmaceutically acceptable salt or solvate thereof.

In one embodiment the present invention provides a method of producing a protein kinase inhibitory effect in a cell or tissue, said method comprising contacting the cell or tissue with an effective amount of a compound, or a pharmaceutically acceptable salt or solvate thereof.

In other embodiment, the present invention provides a method of producing a protein kinase inhibitory effect in vivo, said method comprising administering to a subject an effective amount of a compound, or a pharmaceutically acceptable salt or solvate thereof. The administration may be by any suitable route of administration, such including parenteral or oral administration. The dosage amount may be any suitable amount, for example, the dosage unit for parenteral or oral administration may contain from about 50 mg to about 5000 mg of a compound of Formula I, or Formula II, or a pharmaceutical acceptable salt, or solvate thereof. The compound of the present invention may be administered 1 to 4 times a day. A dosage of between 0.01-100 mg/kg body weight/day of the compound of the present invention can be administered to a patient receiving these compositions.

The compounds of the present invention may be used alone or in combination with one or more other therapeutic agents. The combination may be achieved by way of the simultaneous, sequential or separate dosing of the individual components of treatment. Such combination products employ the compounds of this invention within the dose range described hereinbefore and the other pharmaceutically active agent within its approved dose range.

Another aspect of the present invention provides a method of modulating the target kinase function. The method comprising:

a) contacting a cell with a compound of the present invention in an amount sufficient to modulate the target kinase function, thereby

b) modulating the target kinase activity and signaling.

The present invention further provides a method of synthesis a compound, or a pharmaceutically acceptable salt or solvate thereof, as defined herein.

Another aspect of the present invention provides a probe, the probe comprising a compound of Formula I, or Formula II, labeled with a detectable label or an affinity tag. In other words, the probe comprises a residue of a compound of Formula I, or Formula II, covalently conjugated to a detectable label. Such detectable labels include, but are not limited to, a fluorescent moiety, a chemiluminescent moiety, a paramagnetic contrast agent, a metal chelate, a radioactive isotope-containing moiety, or biotin.

### DETAILED DESCRIPTION OF PREFERRED EMBODIMENTS

The present invention relates to novel kinase inhibitors. These compounds are found to have activity as inhibitors of protein kinases, including members of the Src or Tec kinase families.

Compounds of the present invention may be formulated into a pharmaceutical composition, which comprises an effective amount of a compound of the present invention, with at least one pharmaceutically acceptable diluent, carrier, or excipient.

The term “pharmaceutically effective amount” refers to any amount of the composition for the prevention and treatment of subjects that is effective in treating a disease, disorder, or condition associated with protein kinase activity.

Pharmaceutical Compositions

According to the present invention there is provided a pharmaceutical composition which comprises a compound of Formula I, Formula II, combinations thereof, or a pharmaceutically acceptable salt, solvates, solvates of salts, stereoisomers, tautomers, isotopes, prodrugs, complexes or biologically active metabolites thereof, or mixtures of the compounds of the present invention, in association with at least one pharmaceutically acceptable excipient, diluent, or carrier.

The pharmaceutical compositions may be in a conventional pharmaceutical form suitable for oral administration (e.g., tablet, capsule, granules, powder, liquid solution, suspension or syrup); for parenteral administration (e.g., cutaneous, subcutaneous, intramuscular, intraperitoneal, intravenous, intra-arterial, intra-cerebral, intraocular injection, or infusion); suppository, rectal or vaginal; bronchial, nasal, topical, buccal, sub-lingual, transdermal, or drop infusion preparations, inhalation or insufflations, eye lotion or liquid aerosol. Regardless of the route of administration selected, the compounds may be formulated into pharmaceutically acceptable dosage forms by conventional methods known to those skilled in the art.

In the development of a dosage form formulation, the choice of the core excipients is extremely important. Several aspects of the finished dosage form must be considered such as the nature of the active pharmaceutical ingredient (API), the intended delivery method of the API (immediate release, modified, sustained, extended, delayed release etc), and the manufacturing process.

A non-limiting list of pharmaceutical compositions comprising a compound of Formula I, or Formula II (or combinations of the inventive compounds), according to the present invention, and at least one pharmaceutically acceptable excipient, such as a binder, a disintegrating agent, a lubricant, a diluent, a solubilizing agent, an emulsifier, a coating agent, a cyclodextrin or buffer, for use in formulation of suitable release dosage forms: “prolonged release”, “extended release”, “modified release”, “delayed release”, “sustained release”, or “immediate release”, “orally disintegrating tablets”, or “sustained release parenteral depot” pharmaceutical compositions.

There are different dosage forms with plurality of “controlled release” pharmaceutical compositions, particularly “prolonged release”, “extended release”, “modified release”, “delayed release”, or “sustained release” compositions. Examples for controlled release pharmaceutical compositions are immediate release pharmaceutical compositions, enteric coated pharmaceutical compositions, pulsed release pharmaceutical compositions, or sustained release pharmaceutical compositions.

An oral “controlled release pharmaceutical composition” means a pharmaceutical composition including at least one active pharmaceutical ingredient which is formulated with at least one pharmaceutically acceptable film forming polymer, and optionally with at least one pharmaceutically acceptable excipient, where the pharmaceutical composition shows a pH-dependent. or a pH-independent reproducible release profile.

The term “oral controlled release pharmaceutical composition”, as referred to herein, is defined to mean oral pharmaceutical compositions which when administered releases the active ingredient at a relatively constant rate, and provide plasma concentrations of the active ingredient that remain substantially invariant with time within the therapeutic range of the active ingredient over a 24-hour period, and encompasses “prolonged release”, “extended release”, “modified release”, “delayed release” or “sustained release” compositions.

The term “modified release”, as referred to herein, means that the escape of the drug from the tablet has been modified in some way. Usually, this is to slow the release of the drug so that the medicine doesn't have to be taken too often, and therefore improves compliance. The other benefit from modifying release is that the drug release is controlled, and there are smaller peaks, and troughs in blood levels therefore reducing the chance of peak effects, and increasing the likelihood of therapeutic effectiveness for longer periods of time.

The term “continuous release”, means that a term applied to a drug that is designed to deliver a dose of a medication over an extended period. The most common device for this purpose is a soft, soluble capsule containing minute pellets of the drug for release at different rates in the GI tract, depending on the thickness and nature of the oil, fat, wax, or resin coating on the pellets. Another system consists of a porous plastic carrier, impregnated with the drug, and a surfactant to facilitate the entry of GI fluids that slowly leach out of the drug. Ion exchange resins that bind to drugs and liquids containing suspensions of slow-release drug granules, are also used to provide medication over an extended period.

The term “pulsatile release”, means that a drug is delivered in one, or more doses that fluctuate between a maximum and minimum dose, over a predetermined time intervals. This can be represented by a dose release profile having one or more distinct peaks, or valleys. However, two or more pulsed releases may produce an overlapping, overall, or composite release profile that appears, or effectively is constant. The need for pulsatile release may include the desire to avoid drug degradation in the stomach, or first pass metabolism. Pulsatile release can be achieved via coating of multiparticulates with pH dependent, and/or barrier membrane coating systems, followed by blending of the multiparticulates to achieve desired release profiles.

The term “delayed” release”, refers to the onset of release in relationship to administration of the drug. “Delayed”, means that the release of drug is postponed, and begins, or is triggered some period of time after administration (e.g., the lag time), typically a relatively long period of time, e.g. more than one hour.

The term “immediate release”, means that oral pharmaceutical compositions, which when administered release the active ingredient within a small period of time, typically less than 45 minutes after administration. Oral formulations for immediate release drug delivery system is a conventional type of drug delivery system that designed to disintegrate, and release their pharmaceutically active ingredient with no rate controlling features, such as special coatings and other techniques.

The term “Orally Disintegrating Tablets” (ODT), refers to the tablet that have a disintegration time less than 60 seconds, with good mouth feel and friability that did not exceed 1%. Orally Disintegrating Tablet (ODT) allows to improve patient compliance, in particular with pediatric, geriatric, and institutionalized patients, or patients with chemotherapy-induced nausea.

Oral dosage forms, which may be employed with the present invention include: tablets, granules, spheroids, or pellets in a capsule, or in any other suitable solid form.

A “depot formulation” may be formulated to provide slow absorption of the molecules of Formula I, or Formula II, or combinations thereof, or pharmaceutically acceptable salts, derivatives, isomers, polymorphs, solvates, hydrates, analogues, enantiomers, tautomeric forms, or mixtures thereof from the site of administration, often keeping therapeutic levels of the molecule, or an active metabolite in the patient's system for days or weeks at a time. Alternatively, a depot formulation may provide convenience for a patient in need of chronic medication. By delivering molecules of the present invention without exposure to the GI tract. Moreover, a depot formulation may provide better compliance due to the infrequent dosing regimen and convenience. Additional characteristics of a depot formulation that will enhance patient compliance are good local tolerance at the injection site and ease of administration.

Although the dosage form will vary depending on the symptoms, age, and body weight of the patient, the nature and severity of the disorder to be treated or prevented, the route of administration, and the form of the drug. In general a daily dosage form 0.01 to 2000 mg of the compound is recommended for an adult human patient, and this may be administered in a single dose, or in divided doses. The amount of active ingredient, which can be combined with at least one carrier material, to produce a single dosage form will generally be that amount of the compound which produces a therapeutic effect.

The time of administration, or amount of the composition that will yield the most effective results in terms of efficacy of treatment, in a given patient will depend upon the activity, pharmacokinetics, and bioavailability of a particular compound, physiological condition of the patient (including age, sex, disease type, and stage, general physical condition, responsiveness to a given dosage form, and type of medication), route of administration, etc.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be acceptable in the sense of being compatible with the other ingredients of the formulation, including the active ingredient, and not injurious or harmful to the patient. Some examples of materials which can serve as pharmaceutically acceptable carriers include: (1) sugars, such as lactose, glucose, or sucrose; (2) starches, such as corn starch, potato starch, and substituted or unsubstituted β-cyclodextrin; (3) cellulose, and its derivatives, such as sodium carboxymethyl cellulose, ethyl cellulose, or cellulose acetate; (4) powdered tragacanth; (5) malt; (6) gelatin; (7) talc; (8) excipients, such as cocoa butter or suppository waxes; (9) oils, such as peanut oil, cottonseed oil, safflower oil, sesame oil, olive oil, corn oil, or soybean oil; (10) glycols, such as propylene glycol; (11) polyols, such as glycerin, sorbitol, mannitol, or polyethylene glycol; (12) esters, such as ethyl oleate, or ethyl laurate; (13) agar; (14) buffering agents, such as magnesium hydroxide or aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salt” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like (See, for example, Berge et al. (1977) “Pharmaceutical Salts”, J. Pharm. Sci. 66: 1-19).

The term “halo” or “halogen” refers to chlorine, bromine, fluorine, or iodine. Fluorine is a preferred halogen.

The pharmaceutical compositions of the present invention may be obtained by conventional procedures using conventional pharmaceutical excipients, well known in the art.

In other cases, the compounds of the present invention may contain one or more acidic functional groups and, thus, are capable of forming pharmaceutically acceptable salts with pharmaceutically acceptable bases, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al.).

As used herein, the term “affinity tag”, means a ligand or group, linked either to a compound of the present invention, or to a protein kinase domain, that allows the conjugate to be extracted from a solution.

The term “alkyl”, refers to substituted or unsubstituted saturated hydrocarbon groups, including straight-chain alkyl and branched-chain alkyl groups, including haloalkyl groups such as trifluoromethyl and 2,2,2-trifluoroethyl, etc. Representative alkyl groups include methyl, ethyl, n-propyl, isopropyl, n-butyl, t-butyl, isobutyl, sec-butyl, (cyclohexyl)methyl, cyclopropylmethyl, n-pentyl, n-hexyl, n-heptyl, n-octyl, and the like.

The terms “alkenyl” and “alkynyl”, refer to substituted or unsubstituted unsaturated aliphatic groups analogous in length and possible substitution to the alkyls described above, but that contain at least one double or triple bond respectively. Representative alkenyl groups include vinyl, propen-2-yl, crotyl, isopenten-2-yl, 1,3-butadien-2-yl), 2,4-pentadienyl, and 1,4-pentadien-3-yl. Representative alkynyl groups include ethynyl, 1- and 3-propynyl, and 3-butynyl. In certain preferred embodiments, alkyl substituents are lower alkyl groups, e.g., having from 1 to 6 carbon atoms. Similarly, alkenyl and alkynyl preferably refer to lower alkenyl and alkynyl groups, e.g., having from 2 to 6 carbon atoms. As used herein, “alkylene” refers to an alkyl group with two open valencies (rather than a single valency), such as —(CH₂)₁₋₁₀- and substituted variants thereof.

The term “alkoxy”, refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “alkoxyalkyl”, refers to an alkyl group substituted with an alkoxy group, thereby forming an ether.

The terms “amide” and “amido”, are art-recognized as an amino-substituted carbonyl and includes a moiety that can be represented by the general formula:

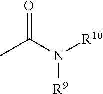

wherein R⁹, R¹⁰ are as defined above. Preferred embodiments of the amide will not include imides, which may be unstable.

The terms “amine” and “amino”. are art-recognized and refer to both unsubstituted and substituted amines and salts thereof, e.g., a moiety that can be represented by the general formulae:

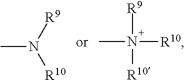

wherein R⁹, R¹⁰ and R^{10′} each independently represent a hydrogen, an alkyl, an alkenyl, —(CH₂)ₚ—R⁸, or R⁹ and R¹⁰ taken together with the N atom to which they are attached complete a heterocycle having from 4 to 8 atoms in the ring structure; R⁸ represents an aryl, a cycloalkyl, a cycloalkenyl, a heterocyclyl, or a polycyclyl; and p is zero or an integer from 1 to 8. In preferred embodiments, only one of R⁹ or R¹⁰ can be a carbonyl, e.g., R⁹, R¹⁰, and the nitrogen together do not form an imide. In even more preferred embodiments, R⁹ and R¹⁰ (and optionally R^{10′}) each independently represent a hydrogen, an alkyl, an alkenyl, or —(CH₂)ₚ—R⁸. In certain embodiments, the amino group is basic, meaning the protonated form has a pKₐ≧7.00.

The term “aralkyl”, as used herein, refers to an alkyl group substituted with an aryl group, for example —(CH₂)ₚ—Ar.

The term “heteroaralkyl”, as used herein, refers to an alkyl group substituted with a heteroaryl group, for example —(CH₂)ₚ—Het.

The term “aryl” as used herein includes 5-, 6-, or 7-membered substituted, or unsubstituted single-ring aromatic groups in which each atom of the ring is carbon. The term “aryl” also includes polycyclic ring systems, having two or more cyclic rings, in which two or more carbons are common to two adjoining rings, wherein at least one of the rings is aromatic, e.g., the other cyclic rings can be cycloalkyls, cycloalkenyls, cycloalkynyls, aryls, heteroaryls, or heterocyclyls. Aryl groups include benzene, naphthalene, phenanthrene, phenol, aniline, anthracene, or phenanthrene.

The terms “carbocycle” and “carbocyclyl”, as used herein, refer to a non-aromatic substituted or unsubstituted ring in which each atom of the ring is carbon. The terms “carbocycle” and “carbocyclyl” also include polycyclic ring systems having two or more cyclic rings, in which two or more carbons are common to two adjoining rings, wherein at least one of the rings is carbocyclic, e.g., the other cyclic rings can be cycloalkyls, cycloalkenyls, cycloalkynyls, aryls, heteroaryls, or heterocyclyls. Representative carbocyclic groups include cyclopentyl, cyclohexyl, 1-cyclohexenyl, or 3-cyclohexen-1-yl, cycloheptyl.

The term “carbonyl” is art-recognized and includes such moieties as can be represented by the general formula:

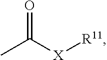

wherein X is a bond or represents an oxygen, or a sulfur, and R¹¹ represents a hydrogen, an alkyl, an alkenyl, —(CH₂)ₚ—R⁸ or a pharmaceutically acceptable salt. Where X is oxygen and R¹¹ is not hydrogen, the formula represents an “ester”. Where X is oxygen, and R¹¹ is hydrogen, the formula represents a “carboxylic acid”.

The terms “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, isoxazole, oxazole, thiazole, triazole, pyrazole, pyridine, pyrazine, pyridazine, or pyrimidine, and the like.

The term “heteroatom” as used herein means an atom of any element other than carbon or hydrogen. Preferred heteroatoms are nitrogen, oxygen, or sulfur.

The terms “heterocyclyl” or “heterocyclic group” refer to substituted or unsubstituted non-aromatic 3- to 10-membered ring structures, more preferably 3- to 7-membered rings, whose ring structures include one to four heteroatoms. The term terms “heterocyclyl” or “heterocyclic group” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, tetrahydrofuran, tetrahydropyran, piperidine, piperazine, pyrrolidine, morpholine, lactones, or lactams.

The term “hydrocarbon”, as used herein, refers to a group that is bonded through a carbon atom that does not have a ═O, or ═S substituent, and typically has at least one carbon-hydrogen bond, and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon), and ethoxy (which is linked through oxygen, not carbon) are not. Hydrocarbyl groups include, but are not limited to aryl, heteroaryl, carbocycle, heterocycle, alkyl, alkenyl, alkynyl, or combinations thereof.

The terms “polycyclyl” or “polycyclic”, refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

As used herein, the term “probe”, means a compound of the invention which is labeled with either a detectable label, or an affinity tag, and which is capable of binding, either covalently or non-covalently, to a protein kinase domain. When, for example, the probe is non-covalently bound, it may be displaced by a test compound. When, for example, the probe is bound covalently, it may be used to form cross-linked adducts, which may be quantified and inhibited by a test compound.

The term “substituted”, refers to moieties having substituents replacing a hydrogen on one or more atoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same, or different for appropriate organic compounds. For purposes of this invention, the heteroatoms such as nitrogen may have hydrogen substituents and/or any permissible substituents of organic compounds described herein, which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Compounds of the invention also include all isotopes of atoms present in the intermediates or final compounds. Isotopes include those atoms having the same atomic number, but different mass numbers. For example, isotopes of hydrogen include deuterium and tritium.

Therapeutic Uses and Applications

The compounds of the present invention are inhibitors of protein kinase activity.

An aspect of the present invention provides a method of inhibiting protein kinase activity in a cell, the method comprising administering to said cell compound of Formula I, or Formula as defined herein, combinations thereof, or a pharmaceutically acceptable salt, or solvate thereof.

In a further aspect, the present invention provides a method of inhibiting protein kinase in vitro or in vivo, said method comprising contacting a cell with an effective amount of a compound, or a pharmaceutically acceptable salt, or solvate thereof, as defined herein.

A further aspect of the present invention provides a method of inhibiting protein kinase activity in a human or animal subject, the method comprising administering to said subject an effective amount of a compound of Formula I, or Formula II, combinations thereof, as defined herein, or a pharmaceutically acceptable salt or solvate thereof.

In one embodiment, the protein kinase is selected from the following group: Tec, Src, Abl, Jak, Csk, Fak, Syk, Fer, Ack kinases, or receptor protein kinases. Preferably the protein kinases are from Tec or Src kinase family. In a particular embodiment the protein kinase is Bruton's tyrosine kinase (Btk).

The compounds of the present invention are suitable for the treatment of diseases or conditions, in which one or more of the protein kinase targets are implicated.

In one embodiment, the compounds are suitable for inhibition of a proliferative disorder mediated by one of the aforementioned protein kinase targets.

In other embodiment, the compounds are suitable for inhibition of a proliferative disorder mediated by Tec kinase targets.

In other embodiment, the compounds are suitable for inhibition of a proliferative disorder mediated by Src kinase targets.

The term “proliferative disorder” is used herein in a broad sense to include any disorder that requires control of deleterious cell proliferation, for example cancers and other disorders associated with uncontrolled cellular proliferation, such as dermatological disorders such as psoriasis, certain viral disorders, certain cardiovascular diseases such as restenosis, or cardiomyopathy, certain CNS disorders, auto-immune disorders such as glomerulonephritis or rheumatoid arthritis, hormone-related diseases, metabolic disorders, stroke, alopecia, emphysema, inflammatory diseases, or infectious diseases such fungal diseases, or parasitic disorders such as malaria. In these disorders, the compounds of the present invention may induce apoptosis, or maintain stasis within the desired cells as required.

The term “protein kinase mediated disease” is used herein associated with abnormal cellular responses triggered by protein kinase-mediated events. Furthermore, aberrant activation or excessive expression of various protein kinases are implicated in the mechanism of multiple diseases and disorders characterized by benign and malignant proliferation. These diseases include, but are not limited to allergies and asthma, Alzheimer's disease, autoimmune diseases, bone diseases, cancer, cardiovascular diseases, inflammatory diseases, hormone-related diseases, metabolic diseases, neurological and neurodegenerative diseases. Thus, inhibitors of kinase families are expected to be suitable in the treatment of cancer, vascular disease, autoimmune diseases, and inflammatory conditions including, but not limited to: solid tumors, hematological malignancies, thrombus, arthritis, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy and dermatomyositis.

In one embodiment, the compound of Formula I, Formula II, combinations thereof, or pharmaceutically acceptable salts, solvates, solvates of salts, stereoisomers, tautomers, isotopes, prodrugs, complexes, or biologically active metabolites thereof, is acting by inhibiting one or more of the host cell kinases involved in cell proliferation, cell survival, viral replication, cardiovascular disorders, neurodegeneration, autoimmunity, a metabolic disorder, stroke, alopecia, an inflammatory disease, or an infectious disease.

In one embodiment, the proliferative disorder is cancer. The cancer may be selected from the group consisting of chronic lymphocytic leukaemia (CLL), lymphoma, leukaemia, breast cancer, lung cancer, prostate cancer, colon cancer, melanoma, pancreatic cancer, ovarian cancer, squamous carcinoma, carcinoma of head or neck, endometrial cancer, or oesophageal carcinoma.

In another embodiment of the present invention, the infectious disease includes diseases that are caused by protozoal infestations in humans or animals. Such veterinary and human pathogenic protozoas are preferably intracellular active parasites of the phylum Apicomplexa, or Sarcomastigophora, especially Trypanosoma, Plasmodia, Leishmania, Babesia, or Theileria, Cryptosporidia, Sacrocystida, Amoebia, Coccidia, or Trichomonadia. The compounds of the present invention are particularly suitable for the treatment of Malaria tropica caused by Plasmodium falciparum, Malaria tertiana caused by Plasmodium vivax, or Plasmodium ovale, or for the treatment of Malaria quartana caused by Plasmodium malariae. These compounds are also suitable for the treatment of Toxoplasmosis caused by Toxoplasma gondii, Coccidiosis caused for instance by Isospora belli, intestinal Sarcosporidiosis caused by Sarcocystis suihominis, dysentery caused by Entamoeba histolytica, Cryptosporidiosis caused by Cryptosporidium parvum, Chagas disease caused by Trypanosoma cruzi, sleeping sickness caused by Trypanosoma brucei, rhodesiense or gambiense, the cutaneous or visceral, as well as other forms of Leishmaniosis. The present invention is also suitable for the treatment of animals infected by veterinary pathogenic Protozoa, like Theileria parva, the pathogen causing bovine East coast fever, Trypanosoma congolense or Trypanosoma vivax, Trypanosoma brucei, pathogens causing Nagana cattle disease in Africa, Trypanosoma brucei evansi causing Surra, Babesia bigemina, the pathogen causing Texas fever in cattle and buffalos, Babesia bovis, the pathogen causing European bovine Babesiosis, as well as Babesiosis in dogs, cats or sheep, Sarcocystis ovicanis or Sarcocystis ovifelis pathogens causing Sarcocystiosis in sheep, cattle or pigs, Cryptosporidia, pathogens causing Cryptosporidioses in cattle and birds, Eimeria or Isospora species, pathogens causing Coccidiosis in rabbits, cattle, sheep, goats, pigs and birds, especially in chickens and turkeys. The compounds of the present invention is particularly preferred for use in the treatment of Coccidiosis or Malaria infections, or for the preparation of a drug, or feed stuff for the treatment of these diseases. These treatments can be prophylactic or curative. In the treatment of malaria, the protein kinase inhibitor, as defined above may be combined with other anti-malaria agents. The present compound described may further be used for viral infections, or other infections caused by Pneumocystis carinii. These compounds may be used alone, or in combination with one, or more agents for the efficient therapy.

Tec kinases is a family of non-receptor tyrosine kinases predominantly, but not exclusively, expressed in cells of hematopoietic origin. The Tec family comprises: Tec, Bruton's tyrosine kinase (Btk), inducible T-cell kinase (Itk), resting lymphocyte kinase (Rlk/Txk), or bone marrow-expressed kinase (Bmx/Etk).

Btk is activated by Src-family kinases and phosphorylates PLC gamma leading to effects on B-cell function and survival. Additionally, Btk is important in signal transduction in response to immune complex recognition by macrophage, mast cells and neutrophils. Btk inhibition is also important in survival of lymphoma cells (Herman SEM. Blood, 2011, 117:6287-6289) suggesting that inhibition of Btk may be useful in the treatment of lymphomas. Bmx, another Tec family member are expected to be suitable in the treatment of various diseases including cancer, cardiovascular disease and inflammation. These compounds may be used alone, or in combination with one or more agents for the therapy.

In further aspect of the present invention, the compound of Formula I, Formula H, combinations thereof, or pharmaceutically acceptable salts, solvates, solvates of salts, stereoisomers, tautomers, isotopes, prodrugs, complexes, or biologically active metabolites thereof, is acting as inhibitor of cell kinases, as anti-inflammatory, anti-cancer, or as antithrombotic agents.

These compounds may be used alone, or in combination with one or more agents, for the treatment of cancer, inflammatory or infectious diseases, or thrombi.

More specifically, the compounds of the present invention can be used in combination with at least one chemotherapeutic agent for use particularly in treatment of cancer, neoplasms, or other proliferative diseases or disorder.

The compounds of Formula I, Formula II, combinations thereof, or pharmaceutically acceptable salts, solvates, solvates of salts, stereoisomers, tautomers, isotopes, prodrugs, complexes or biologically active metabolites thereof, can be used in combination with, but not limiting to:
1. Anti-proliferative agents, selected from the group of: adriamycin, dexamethasone, vincristine, cyclophosphamide, fluorouracil, topotecan, taxol, interferons, platinum derivatives; anti-inflammatory agents comprising corticosteroids, TNF blockers, IL-1 RA, azathioprine, cyclophosphamide, or sulfasalazine;
2. Prenyl-protein transferase inhibitors;
3. Angiogensis inhibitors, comprising: sorafenib, sunitinib, pazopanib, or everolimus;
4. Immunomodulatory or immunosuppressive agents selected from the group comprising: cyclosporin, tacrolimus, rapamycin, mycophenolate mofetil, interferons, corticosteroids, cyclophophamide, azathioprine, or sulfasalazine;
5. PPAR-γ agonists such as thiazolidinediones;
6. PPAR-δ agonists;
7. Inhibitors of inherent multidrug resistance;
8. Agents for the treatment of anemia, comprising erythropoiesis, stimulating agents, vitamins, or iron supplements;
9. Anti-emetic agents including: 5-HT3 receptor antagonists, dopamine antagonists, NK1 receptor antagonists, H1 histamine receptor antagonists, cannabinoids, benzodiazepines, anticholinergic agents, or steroids;
10. Agents for the treatment of neutropenia;
11. Immunologic-enhancing agents;
12. Proteasome inhibitors;
13. HDAC inhibitors;
14. Inhibitors of the chemotrypsin-like activity in the proteasome;
15. E3 ligase inhibitors;
16. Modulators of the immune system including: interferon-alpha, Bacillus Calmette-Guerin (BCG), or ionizing radition (UVB) that can induce the release of cytokines, such as the interleukins, TNF, or induce release of death receptor ligands such as TRAIL;
17. Modulators of death receptors TRAIL or TRAIL-agonists, including humanized antibodies HGS-ETR1, or HGS-ETR in combination, or sequentially with radiation therapy;
18. Neurotrophic factors comprising: acetylcholinesterase inhibitors, MAO inhibitors, interferons, anti-convulsants, ion channel blockers, or riluzole;
19. Anti-Parkinsonian agents comprising: anticholinergic agents, dopaminergic agents, including dopaminergic precursors, monoamine oxidase B inhibitors, COMT inhibitors, or dopamine receptor agonists;
20. Agents for treating cardiovascular disease comprising: beta-blockers, ACE inhibitors, diuretics, nitrates, calcium channel blockers, or statins;
21. Agents for treating liver disease comprising: corticosteroids, cholestyramine, or interferons;
22. Anti-viral agents including: nucleoside reverse transcriptase inhibitors, nonnucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry Inhibitors;
23. Agents for treating blood disorders including: corticosteroids, anti-leukemic agents, or growth factors;
24. Agents for treating immunodeficiency disorders comprising: gamma globulin, adalimumab, etarnecept, or infliximab; or
25. HMG-CoA reductase inhibitors comprising: torvastatin, fluvastatin, lovastatin, pravastatin, rosuvastatin, simvastatin, or pitavastatin.

As defined herein an effect against a proliferative disorder mediated by a kinase within the scope of the present invention may be demonstrated by the ability to inhibit a purified kinase in vitro or to inhibit cell proliferation or survival in an in vitro cell assay, for example in Btk Kinase Inhibition Assay and Splenic Cell Proliferation Assay. These assays are described in more details in the accompany examples.

The present invention includes the transdermal, rectal, parenteral, or oral administration of compounds of Formula I, or Formula II (or combinations thereof) to a human or animal subject. The dosage unit may contain any suitable amount a compound of Formula I, Formula II, combinations thereof (or a pharmaceutical acceptable salt or solvate thereof, or combinations thereof), for example from about 10 mg to about 5000 mg. Preferably, the dosage unit for oral administration may contain from 50 mg to 500 mg, per human individual condition.

The compound of the present invention may be administered 1 to 4 times a day. A dosage may be between 0.01-100 mg/kg body weight/day of the compound of the present invention may be administered to a patient receiving these compositions. The dose can vary within wide limits and is to be suited to the individual conditions in each individual case. For the above uses the appropriate dosage will vary depending on the mode of administration, the particular condition to be treated and the effect desired. Preferably a dose of 1 to 50 mg/kg body weight/day may be used.

In an embodiment of the present invention suitable dosage rates for larger mammals, for example humans, are of the order of from about 10 mg to 3 g/day, administered orally once, or divided doses, such as 2 to 4 times a day, or in sustained release form. For topical delivery, depending on the permeability of the skin, the type and the severity of the disease and dependent on the type of formulation and frequency of application, different concentrations of active compounds within the medicament can be sufficient to elicit a therapeutic effect by topical application. Preferably, the concentration of an active compound pharmaceutically acceptable salts, solvates, solvates of salts, stereoisomers, tautomers, isotopes, prodrugs, complexes or biologically active metabolites thereof, within a medicament according to the present invention is in the range of between 1 μmol/L and 100 mmol/L.

### SPECIFIC ABBREVIATIONS

MS mass spectrometry

ml milliliter

μl microliter

mmol millimole

THF tetrahydrofuran

DMF dimethylformamide

DMSO dimethyl sulfoxide

MeOH methanol

HCl hydrogen chloride

NaH sodium hydride (60% in mineral oil)

CuI copper (I) iodide

Cs₂CO₃ cesium carbonate

K₂CO₃ potassium carbonate

DIPEA N,N-diisopropylethylamine

TEA triethylamine

MgSO₄ magnesium sulfate

NaHCO₃ sodium bicarbonate

NH₄OH ammonium hydroxide

iPrOH isopropyl alcohol

NBS N-bromosuccinimide

NIS N-iodosuccinimide

BBr₃ boron tribromide

PPTS pyridinium p-toluenesulfonate

NaBH₄ sodium borohydride

NaBH(OAc)₃ sodium triacethoxyborohydride

NaOH sodium hydroxide

Ac₂O acetic anhydride

TFA trifluoroacetic acid

DIBALH diisobuthylaluminium hydride

DME ethylene glycol dimethyl ether

DIAD diisopropyl azodicarboxylate

CaCl₂ calcium chloride

(Cy)₃P triclyclohexylphosphine

PPh₃ triphenyl phosphine

PdCl₂(dppf) [1,1′-Bis(diphenylphosphino)ferrocene]dichloropalladium(II)

Pd₂(dba)₃ Tris(dibenzylideneacetone)dipalladium(0)

General Synthetic Methods

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

In further embodiment of the present invention is provided general synthetic method(s) useful in the preparation of compounds described in the present invention.

General Synthetic Method A:

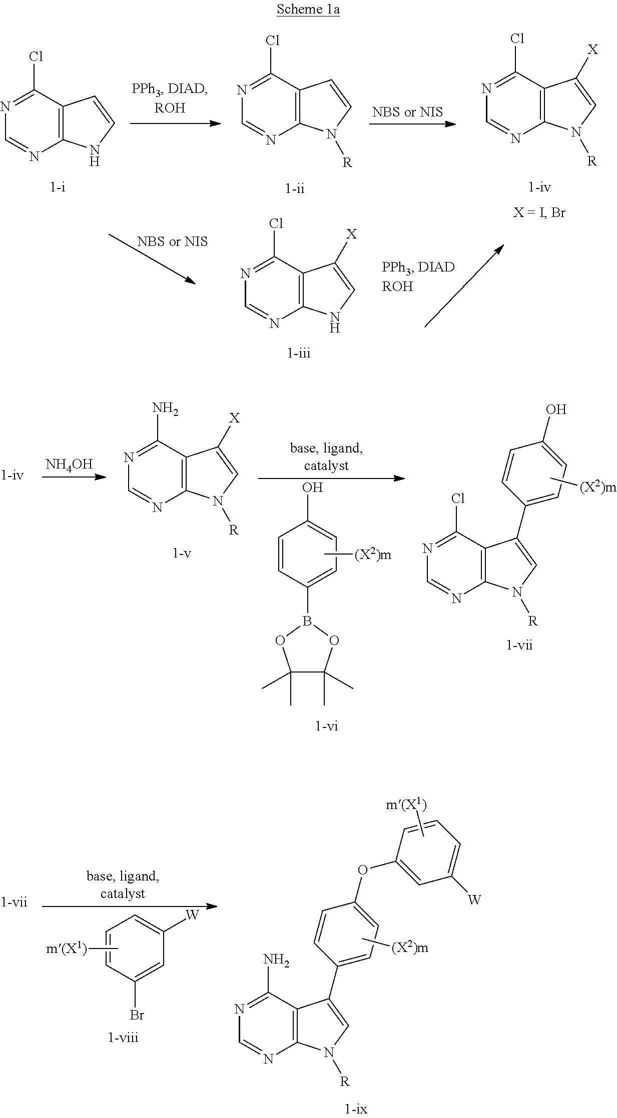

General Synthetic Method B:

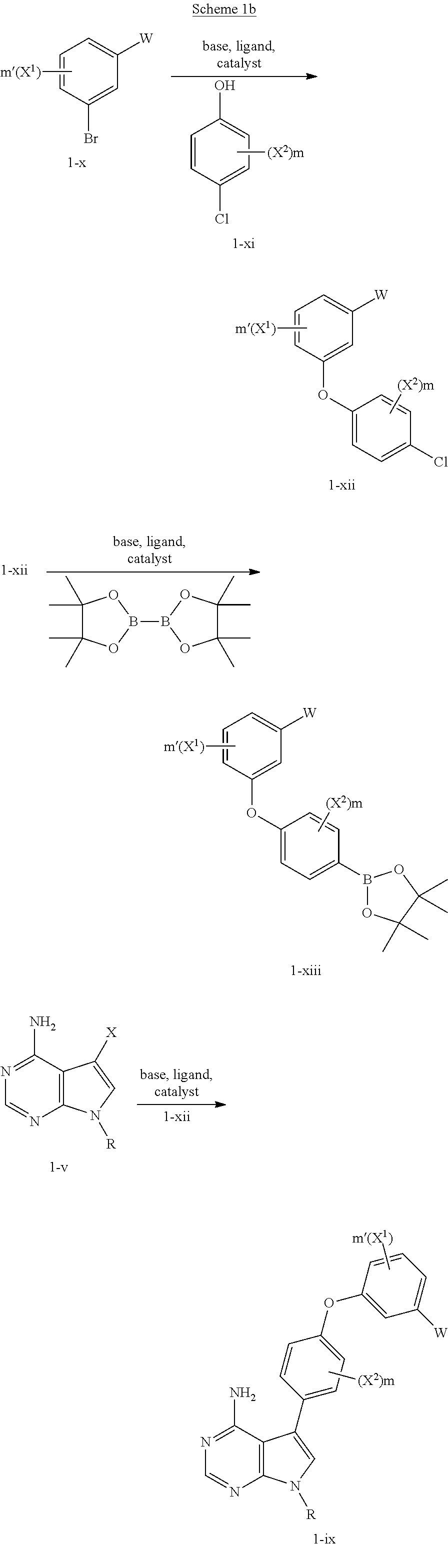

### EXAMPLES

The following synthetic methods are intended to be representative of the chemistry used to prepare compounds of the present invention and are not intended to be limiting.

### Synthesis of Intermediate 2-c

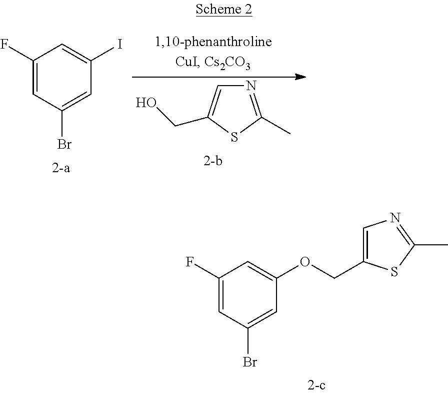

To a solution of 1-bromo-3-fluoro-5-iodobenzene 2-a (7.5 g, 25.0 mmol) in 1,4-dioxane (12.5 ml) was added (2-methylthiazol-5-yl)methanol 2-b (3.5 g, 27.5 mmol), 1,10-phenanthroline (901 mg, 5.0 mmol), copper (I) iodide (476 mg, 2.50 mmol), and cesium carbonate (11.40 g, 35.0 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered, and concentrated under reduced pressure. Purification by silica gel chromatography provided Intermediate 2-c as a beige oil which solidify upon standing.

### Synthesis of Intermediate 3-b

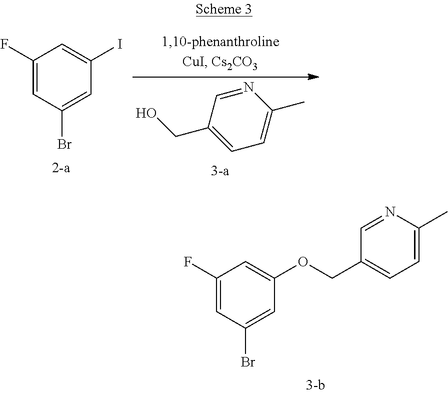

To a solution of 1-bromo-3-fluoro-5-iodobenzene 2-a (5.0 g, 16.62 mmol) in toluene (8.3 ml) was added (6-methylpyridin-3-yl) methanol 3-a (2.25 g, 18.28 mmol), 1,10-phenanthroline (599 mg, 3.32 mmol), copper (I) iodide (316 mg, 1.66 mmol), and cesium carbonate (7.58 g, 23.26 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered, and concentrated under reduced pressure. Purification by silica gel chromatography provided Intermediate 3-b as a beige solid.

### Synthesis of Intermediate 4-b

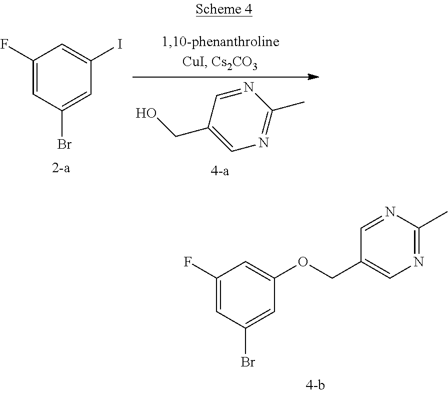

To a solution of 1-bromo-3-fluoro-5-iodobenzene 2-a (5.0 g, 16.62 mmol) in toluene (8.3 ml) was added (2-methylpyrimidin-5-yl)methanol 4-a (2.26 g, 18.28 mmol), 1,10-phenanthroline (599 mg, 3.32 mmol), copper (I) iodide (316 mg, 1.66 mmol), and cesium carbonate (7.58 g, 23.26 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered, and concentrated under reduced pressure. Purification by silica gel chromatography provided Intermediate 4-b as a beige solid.

### Synthesis of Intermediate 5-d

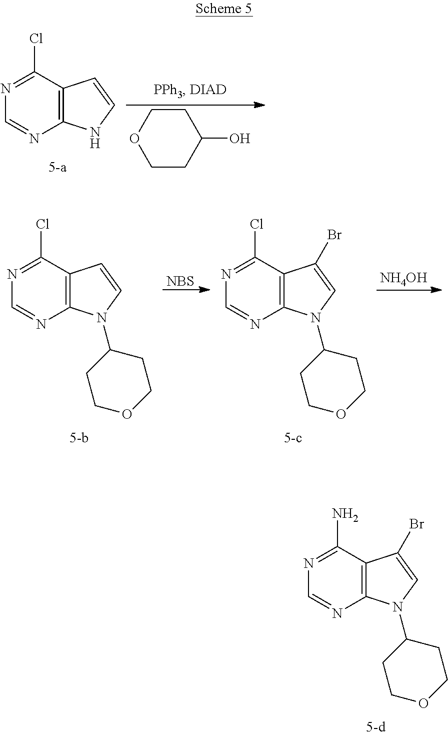

### Step 1: Intermediate 5-b

To a solution of 4-chloro-7H-pyrrolo[2,3-d]pyrimidine 5-a (3.0 g, 19.54 mmol) and tetrahydro-2H-pyran-4-ol (2.99 g, 29.3 mmol) in THF (150 mL) were sequentially added triphenylphosphine (6.7 g, 25.4 mmol), and DIAD (4.9 ml, 25.4 mmol). The solution was stirred at room temperature overnight. Volatiles were removed under reduced pressure. Purification by silica gel chromatography provided Intermediate 5-b as a beige gum.

### Step 2: Intermediate 5-c

To a solution of Intermediate 5-b (2.5 g, 10.5 mmol) in DMF (26.3 ml) cooled to 0° C., was slowly added a 0.7N solution of N-bromosuccinimide in DMF (16.5 ml, 11.5 mmol). The reaction mixture was stirred for 15 minutes at 0° C. Water (70 mL) was added; a precipitate formed, and was collected by filtration to provide Intermediate 5-c as a beige solid.

### Step 3: Intermediate 5-d

To a solution of Intermediate 5-c (2.6 g, 8.2 mmol) in iPrOH (41.4 ml) was added ammonium hydroxide (56.0 ml). The reaction mixture was stirred for 36 hours, at 90° C., and then cooled to room temperature. Volatiles were removed under reduced pressure. The residue was triturated in water; a precipitated formed and was collected by filtration to provide Intermediate 5-d as a beige solid.

### Synthesis of intermediate 6-c

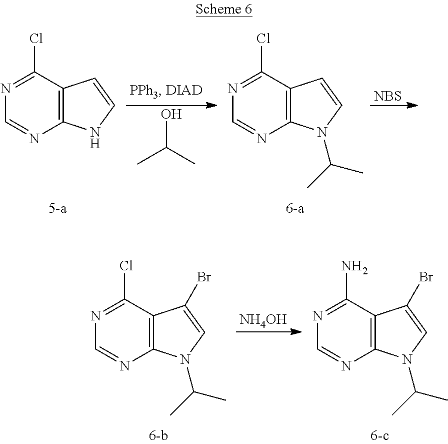

### Step 1: Intermediate 6-a

To a solution of 4-chloro-7H-pyrrolo[2,3-d]pyrimidine 5-a (3.0 g, 19.54 mmol) and 2-propanol (1.5 g, 26.0 mmol) in THF (100 mL) were sequentially added triphenylphosphine (4.4 g, 16.9 mmol), and DIAD (3.3 ml, 16.9 mmol), and the solution was then stirred at room temperature overnight. Volatiles were removed under reduced pressure. Purification by silica gel chromatography provided Intermediate 6-a as a beige gum.

### Step 2: Intermediate 6-b

To a solution of Intermediate 6-a (2.1 g, 10.7 mmol) in DMF (26.8 ml) cooled to 0° C., was slowly added a 0.7N solution of N-bromosuccinimide in DMF (16.8 ml, 11.8 mmol). The reaction mixture was stirred for 15 minutes at 0° C. Water (70 mL) was added; a precipitate formed, and was collected by filtration to provide Intermediate 6-b as a beige solid.

### Step 3: Intermediate 6-c

To a solution of Intermediate 6-b (2.6 g, 9.2 mmol) in iPrOH (12.9 ml) was added ammonium hydroxide (18.0 ml). The reaction mixture was stirred overnight at 90° C., and then cooled to room temperature. Volatiles were removed under reduced pressure. The residue was triturated in water; a precipitated formed, and was collected by filtration to provide Intermediate 6-c as a beige solid.

### Synthesis of Intermediate 7-a

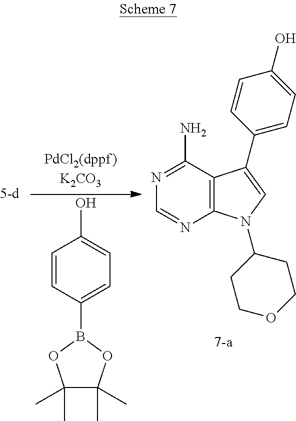

To a solution of Intermediate 5-d (2.3 g, 7.7 mmol) in DME (48 ml) were added potassium carbonate (3.3 g, 23.9 mmol), water (11.9 ml), and 4-(4,4,5,5-tetramethyl-1,3,2-dioxaborolan-2-yl)phenol (1.9 g, 8.9 mmol). The mixture was degassed and PdCl₂(dppf) (428 mg, 0.6 mmol) was added under nitrogen. The reaction mixture was stirred for 2 days at 90° C., and then cooled to room temperature. Volatiles were removed under reduced pressure. Purification by silica gel chromatography provided Intermediate 7-a as a brown solid.

### Synthesis of Intermediate 8-a

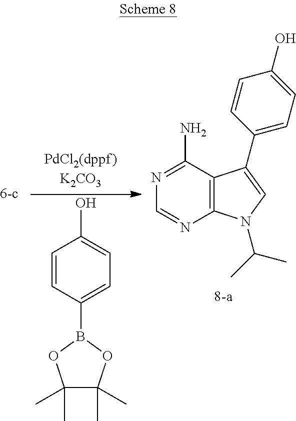

To a solution of Intermediate 6-c (2.4 g, 9.4 mmol) in DME (58 ml) were added potassium carbonate (4.0 g, 29.2 mmol), water (14.5 ml), and 4-(4,4,5,5-tetramethyl-1,3,2-dioxaborolan-2-yl)phenol (2.4 g, 10.8 mmol). The mixture was degassed and PdCl₂(dppf) (347 mg, 0.5 mmol) was added under nitrogen. The reaction mixture was stirred overnight at 90° C., and then cooled to room temperature. Volatiles were removed under reduced pressure. Purification by silica gel chromatography provided Intermediate 8-a as a brown solid.

### Synthesis of Compound 5

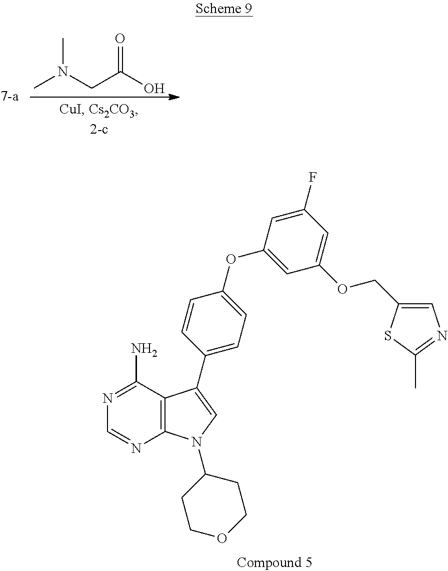

A solution of Intermediate 7-a (210 mg, 0.7 mmol), Intermediate 2-c (245 mg, 0.8 mmol), N,N-Dimethylglycine (209 mg, 2.0 mmol), cesium carbonate (882 mg, 2.7 mmol), and copper(I) iodide (129 mg, 0.7 mmol) in 1,4-dioxane (1.0 ml) was heated in a pressure vessel at 110° C. for 36 hours, then cooled to room temperature. Ethyl acetate was added; the reaction was filtered over celite and adsorbed on silica gel. Purification by silica gel chromatography provided Compound 5 as a beige solid. MS (m/z) M+H=532.3

### Synthesis of Compound 1

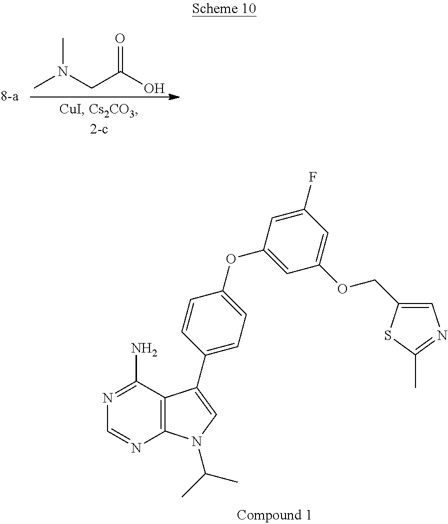

A solution of Intermediate 8-a (200 mg, 0.7 mmol), Intermediate 2-c (270 mg, 0.9 mmol), N,N-Dimethylglycine (115 mg, 1.2 mmol), cesium carbonate (729 mg, 2.2 mmol), and copper(I) iodide (71 mg, 0.4 mmol) in 1,4-dioxane (1.0 ml) was heated in a pressure vessel at 110° C. for 36 hours, and then cooled to room temperature. Ethyl acetate was added; the reaction was filtered over celite, and adsorbed on silica gel. Purification by silica gel chromatography provided Compound 1 as a beige solid. MS (m/z) M+H=490.2

### Synthesis of Compound 4

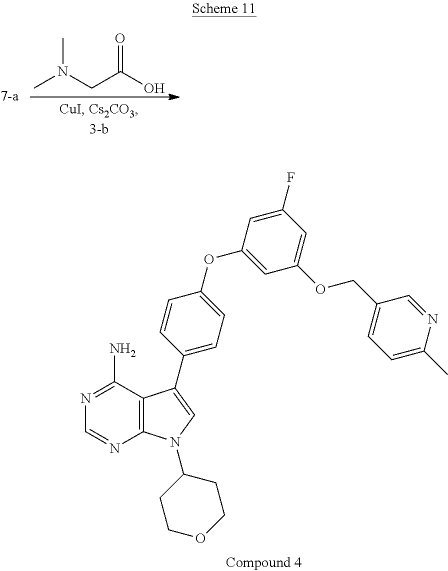

A solution of Intermediate 7-a (210 mg, 0.7 mmol), Intermediate 3-b (240 mg, 0.8 mmol), N,N-Dimethylglycine (209 mg, 2.0 mmol), cesium carbonate (882 mg, 2.7 mmol), and copper(I) iodide (129 mg, 0.7 mmol) in 1,4-dioxane (1.0 ml) was heated in a pressure vessel at 110° C. for 36 hours, then cooled to room temperature. Ethyl acetate was added, the reaction was filtered over celite, and adsorbed on silica gel. Purification by silica gel chromatography provided Compound 4 as a beige solid. MS (m/z) M+H=526.3

### Synthesis of Compound 2

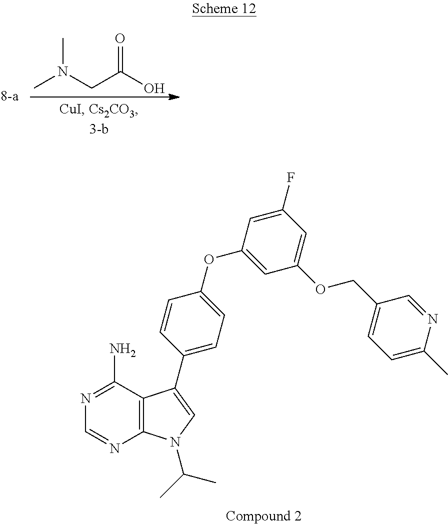

A solution of Intermediate 8-a (200 mg, 0.7 mmol), Intermediate 3-b (265 mg, 0.9 mmol), N,N-Dimethylglycine (115 mg, 1.2 mmol), cesium carbonate (729 mg, 2.2 mmol), and copper(I) iodide (71 mg, 0.4 mmol) in 1,4-dioxane (1.0 ml) was heated in a pressure vessel at 110° C. for 36 hours, then cooled to room temperature. Ethyl acetate was added, the reaction was filtered over celite, and adsorbed on silica gel. Purification by silica gel chromatography provided Compound 2 as a beige solid. MS (m/z) M+H=484.2

### Synthesis of Compound 6

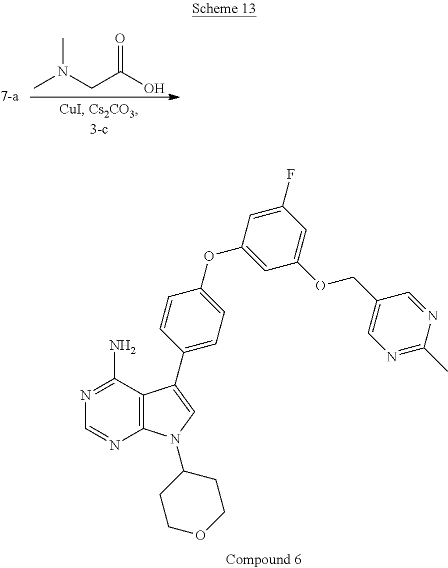

A solution of Intermediate 7-a (210 mg, 0.7 mmol), Intermediate 3-c (241 mg, 0.8 mmol), N,N-Dimethylglycine (209 mg, 2.0 mmol), cesium carbonate (882 mg, 2.7 mmol), and copper(I) iodide (129 mg, 0.7 mmol) in 1,4-dioxane (1.0 ml) was heated in a pressure vessel at 110° C. for 36 hours, and then cooled to room temperature. Ethyl acetate was added, the reaction was filtered over celite, and adsorbed on silica gel. Purification by silica gel chromatography provided Compound 6 as a beige solid. MS (m/z) M+H=527.2

### Synthesis of Compound 3

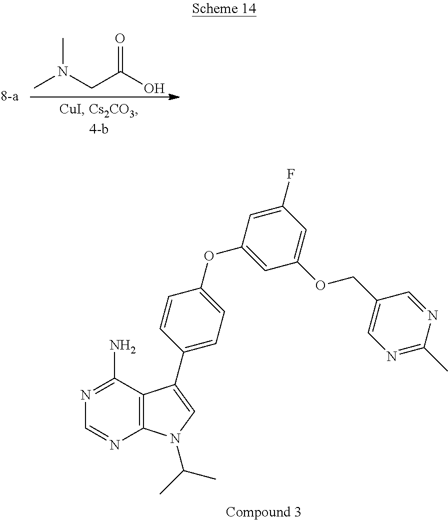

A solution of Intermediate 8-a (200 mg, 0.7 mmol), Intermediate 4-b (266 mg, 0.9 mmol), N,N-Dimethylglycine (115 mg, 1.2 mmol), cesium carbonate (729 mg, 2.2 mmol), and copper(I) iodide (71 mg, 0.4 mmol) in 1,4-dioxane (1.0 ml) was heated in a pressure vessel at 110° C. for 36 hours, and then cooled to room temperature. Ethyl acetate was added, the reaction was filtered over celite, and adsorbed on silica gel. Purification by silica gel chromatography provided Compound 3 as a beige solid. MS (m/z) M+H=485.2

Compound 17 is obtained in a similar manner to Compound 10 starting from commercially available starting materials.

### Synthesis of Intermediate 15-b

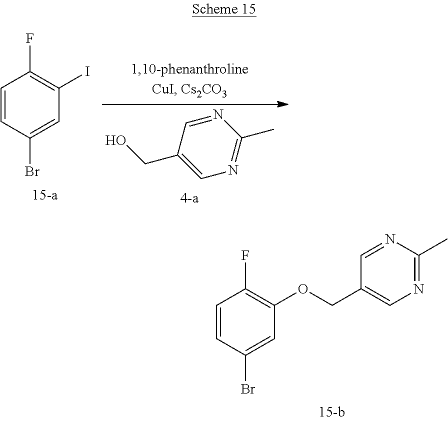

To a solution of 1-fluoro-3-bromo-2-iodobenzene 15-a (5.0 g, 15.4 mmol) in toluene (5.4 ml) was added (2-methylpyrimidin-5-yl)methanol 4-a (1.5 g, 12.1 mmol), 1,10-phenanthroline (396 mg, 2.2 mmol), copper (I) iodide (209 mg, 1.1 mmol), and cesium carbonate (5.0 g, 15.4 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered and concentrated under reduced pressure. Purification by silica gel chromatography provided Intermediate 15-b as a yellow oil.

### Synthesis of Intermediate 16-b

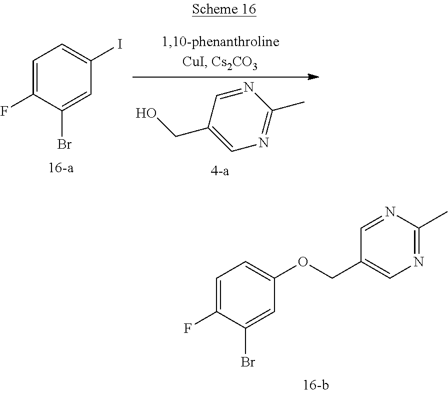

To a solution of 2-bromo-1-fluoro-4-iodobenzene 16-a (3.3 g, 11.0 mmol) in toluene (5.5 ml) was added (2-methylpyrimidin-5-yl)methanol 4-a (1.5 g, 12.1 mmol), 1,10-phenanthroline (396 mg, 2.2 mmol), copper (I) iodide (209 mg, 1.1 mmol), and cesium carbonate (5.0 g, 15.4 mmol). The reaction was stirred at 110° C. for 2 days, and then cooled to room temperature, diluted with ethyl acetate, and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered, and concentrated under reduced pressure. Purification by silica gel chromatography provided Intermediate 16-b as a yellow solid.

### Synthesis of Intermediate 17-b

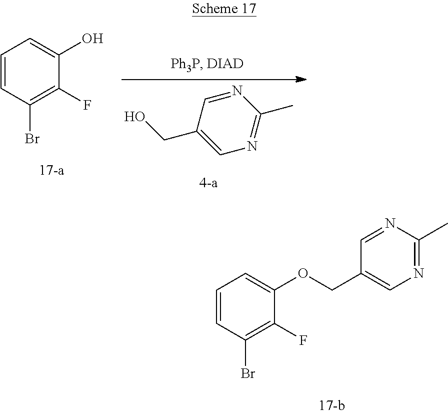

To a solution of 3-bromo-2-fluorophenol 17-a (750 mg, 3.9 mmol) and (2-methylpyrimidin-5-yl)methanol 4-a (487 mg, 3.9 mmol) in THF (3.9 ml) were sequentially added triphenylphosphine (1.5 g, 5.9 mmol), and DIAD (1.2 ml, 6.3 mmol). The reaction was then stirred at room temperature overnight. Volatiles were removed under reduced pressure. Purification by silica gel chromatography provided Intermediate 17-b as a beige solid.

### Synthesis of Compound 15

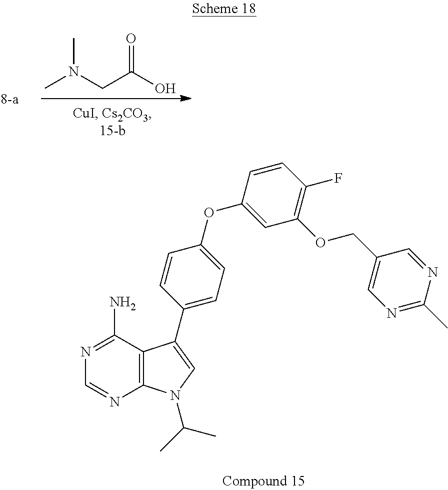

A solution of Intermediate 8-a (100 mg, 0.4 mmol), intermediate 15-b (111 mg, 0.4 mmol), N,N-Dimethylglycine (115 mg, 1.2 mmol), cesium carbonate (486 mg, 1.5 mmol), and copper(I) iodide (71 mg, 0.4 mmol) in 1,4-dioxane (1.0 ml) was heated in a pressure vessel at 110° C. for 2 days, and then cooled to room temperature. Ethyl acetate was added; the reaction was filtered over celite, and adsorbed on silica gel. Purification by silica gel chromatography provided Compound 15 as white solid. MS (m/z) M+H=485.1

### Synthesis of Compound 11

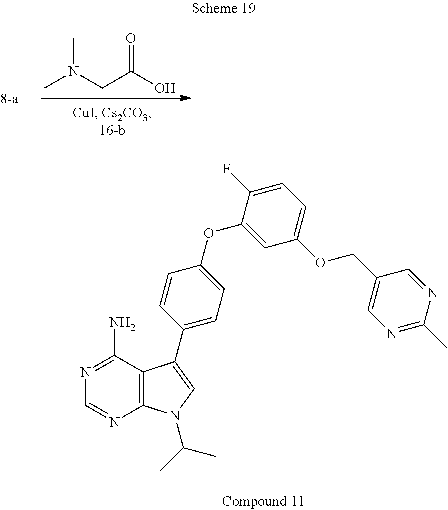

A solution of Intermediate 8-a (100 mg, 0.4 mmol), intermediate 16-b (111 mg, 0.4 mmol), N,N-Dimethylglycine (115 mg, 1.2 mmol), cesium carbonate (486 mg, 1.5 mmol), and copper(I) iodide (71 mg, 0.4 mmol) in 1,4-dioxane (1.0 ml) was heated in a pressure vessel at 110° C. for 2 days, and then cooled to room temperature. Ethyl acetate was added; the reaction was filtered over celite, and adsorbed on silica gel. Purification by silica gel chromatography provided Compound 11 as white solid. MS (m/z) M+H=485.1

### Synthesis of Compound 19

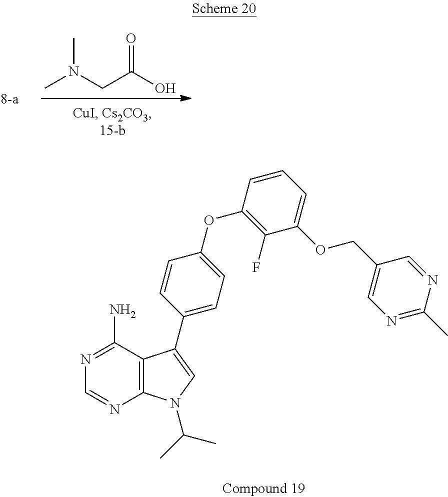

A solution of Intermediate 8-a (72 mg, 0.3 mmol), intermediate 17-b (80 mg, 0.3 mmol), N,N-Dimethylglycine (83 mg, 0.8 mmol), cesium carbonate (351 mg, 1.1 mmol), and copper(I) iodide (51 mg, 0.3 mmol) in 1,4-dioxane (0.6 ml) was heated in a pressure vessel at 110° C. for 2 days, and then cooled to room temperature. Ethyl acetate was added; the reaction was filtered over celite, and adsorbed on silica gel. Purification by silica gel chromatography provided Compound 19 as white foam. MS (m/z) M+H=485.1

### Synthesis of Intermediate 21-f

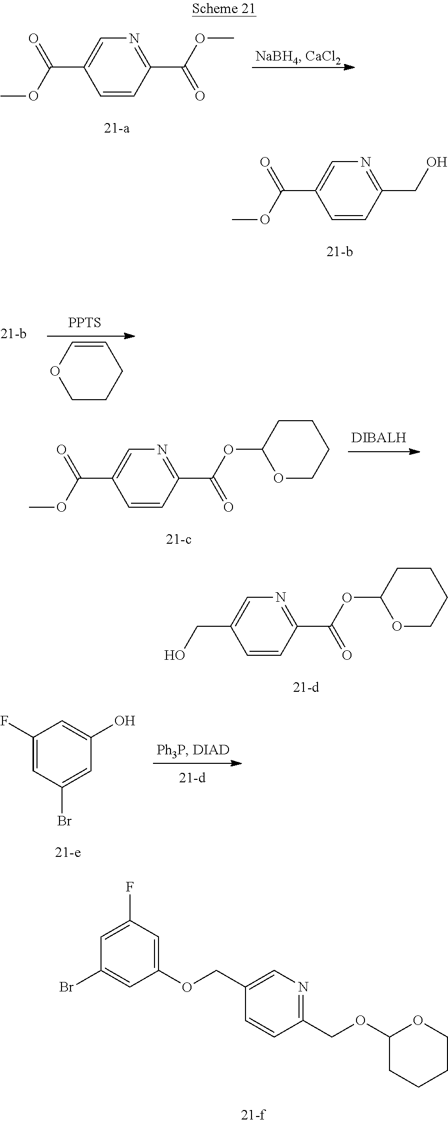

### Step 1: Intermediate 21-b

To a solution of dimethyl pyridine-2,5-dicarboxylate 21-a (13.0 g, 66.6 mmol) in a mixture of THF (110 mL) and ethanol (110 mL) was added calcium chloride (29.6 g, 266 mmol). After stirring at room temperature for 30 minutes, the reaction was cooled to 0° C., and sodium borohydride (3.78 g, 100 mmol) was added portion wise. After the addition was completed the reaction was stirred at room temperature overnight. A saturated aqueous solution of ammonium chloride and dichloromethane were added, the organic layer was separated, and the aqueous phase was extracted twice with dichloromethane. The combined organic extracts were washed with brine, dried over MgSO₄, filtered, and concentrated under reduced pressure to provide Intermediate 21-b as a yellow solid.

### Step 2: Intermediate 21-c

To a solution of Intermediate 21-b (1.70 g, 10.17 mmol) n dichloromethane (203 mL) was added 3,4-dihydro-2H-pyran (4.28 g, 50.8 mmol) and PPTS (2.56 g, 10.17 mmol) and the reaction was stirred at room temperature overnight. Water was added and the organic layer was separated, washed with brine, dried over MgSO₄, filtered, and concentrated under reduced pressure to provide Intermediate 21-c as a white solid.

### Step 3: Intermediate 21-d

To a solution of Intermediate 21-c (2.56 g, 10.17 mmol) in THF (51 ml) cooled to 0° C. was added dropwise a 1.0 M solution of DIBALH in hexane (23.39 ml, 23.39 mmol), and the reaction was then stirred at 0° C. for 1.5 hour, and room temperature overnight. Water (1.0 ml) was slowly added, followed 15% NaOH (3.5 ml), and water (2.3 ml), and the mixture was stirred at room temperature for 30 minutes. The reaction was filtered over celite, and volatiles were removed under reduced pressure. Purification by silica gel chromatography provided Intermediate 21-d as a yellow oil.

### Step 4: Intermediate 21-f

To a solution of Intermediate 3-bromo-5-fluorophenol 21-e (2.5 g, 13.2 mmol) and intermediate 21-d (3.2 g, 14.5 mmol) in THF (13.2 ml) were sequentially added triphenylphosphine (5.2 g, 19.7 mmol), and DIAD (4.26 g, 21.1 mmol) at room temperature, and the reaction was then stirred overnight. Volatiles were removed under reduced pressure. Purification by silica gel chromatography provided Intermediate 21-f as a yellow oil.

### Synthesis of Compound 21

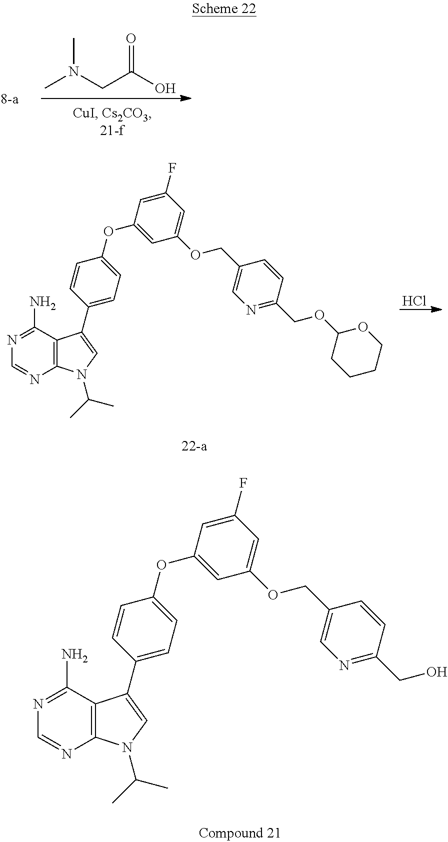

### Step 1: Intermediate 22-a

A solution of Intermediate 8-a (125 mg, 0.5 mmol), intermediate 21-f (203 mg, 0.5 mmol), N,N-Dimethylglycine (144 mg, 1.4 mmol), cesium carbonate (607 mg, 1.9 mmol) and copper(I) iodide (89 mg, 0.5 mmol) in 1,4-dioxane (1.1 ml) was heated in a pressure vessel at 110° C. for 2 days, and then cooled to room temperature. Ethyl acetate was added; the reaction was filtered over celite, and adsorbed on silica gel. Purification by silica gel chromatography provided Intermediate 22-a as a beige oil.

### Step 2: Compound 21

To a solution of Intermediate 22-a (24 mg, 0.04 mmol) in MeOH (1.6 ml) was added 3N HCl (0.9 ml, 2.9 mmol) and the reaction was stirred at room temperature for 1 hour. Volatiles were removed under reduced pressure. Purification by silica gel chromatography eluting with a 0.1% HCl/methanol gradient provided Compound 21.2HCl as a white solid. MS (m/z) M+H=500.2

### Synthesis of Intermediate 23-d

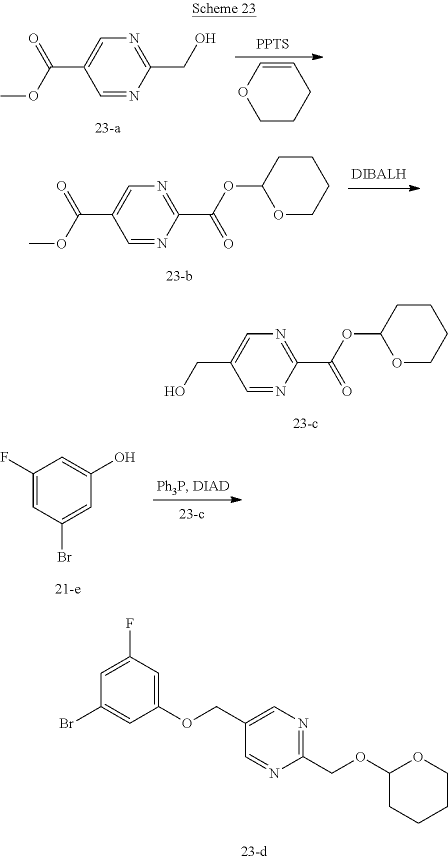

### Step 1: Intermediate 23-b

To a solution of Intermediate 23-a (500 mg, 2.9 mmol) in dichloromethane (60 mL) was added 3,4-dihydro-2H-pyran (1.3 g, 14.9 mmol), and PPTS (747 mg, 2.9 mmol), and the reaction was stirred at room temperature overnight. Water was added and the organic layer was separated, washed with brine, dried over MgSO₄, filtered, and concentrated under reduced pressure to provide Intermediate 23-b as a white solid.

### Step 2: Intermediate 23-c

To a solution of Intermediate 23-b (730 mg, 2.9 mmol) in THF (14 ml) cooled to 0° C. was added dropwise a 1.0 M solution of DIBALH in hexane (12.1 ml, 12.1 mmol), and the reaction was then stirred at 0° C. for 1.5 hour, and room temperature overnight. Water (0.5 ml) was slowly added, followed 15% NaOH (0.5 ml), and water (1.2 ml), and the mixture was stirred at room temperature for 30 minutes. The reaction was filtered over celite, and volatiles were removed under reduced pressure. Purification by silica gel chromatography provided Intermediate 23-c as a yellow oil.

### Step 3: Intermediate 23-d

To a solution of Intermediate 3-bromo-5-fluorophenol 21-e (170 mg, 0.9 mmol) and intermediate 23-c (200 mg, 0.9 mmol) in THF (1.0 ml) were sequentially added triphenylphosphine (351 mg, 19.7 mmol), and DIAD (277 μl, 1.4 mmol) at room temperature, and the reaction was then stirred overnight. Volatiles were removed under reduced pressure. Purification by silica gel chromatography provided Intermediate 23-d as a white solid.

### Synthesis of Compound 20

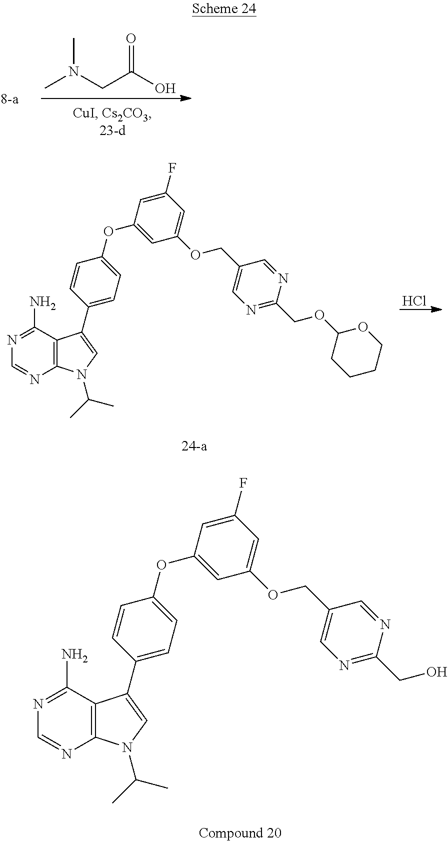

### Step 1: Intermediate 24-a

A solution of Intermediate 8-a (215 mg, 0.8 mmol), Intermediate 23-d (350 mg, 0.9 mmol), N,N-Dimethylglycine (248 mg, 2.4 mmol), cesium carbonate (1.0 g, 3.2 mmol), and copper(I) iodide (143 mg, 0.8 mmol) in 1,4-dioxane (2.0 ml) was heated in a pressure vessel at 110° C. for 2 days, and then cooled to room temperature. Ethyl acetate was added; the reaction was filtered over celite, and adsorbed on silica gel. Purification by silica gel chromatography provided intermediate 24-a as a beige oil.

### Step 2: Compound 20

To a solution of Intermediate 24-a (39 mg, 0.07 mmol) in MeOH (2.6 ml) was added 3N HCl (1.6 ml, 4.7 mmol), and the reaction was stirred at room temperature for 1 hour. Volatiles were removed under reduced pressure. Purification by silica gel chromatography eluting with a 0.1% HCl/methanol gradient provided Compound 20.2HCl as a beige solid. MS (m/z) M+H=501.1

### Synthesis of Intermediate 25-e

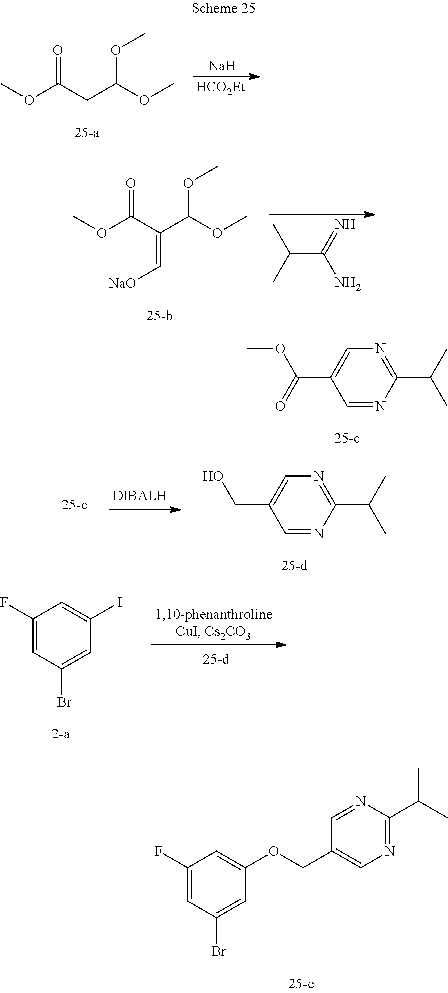

### Step 1: Intermediate 25-b

To a solution of 3,3-dimethoxypropionate 25-a (2.4 ml, 16.9 mmol) in dry DME (12.0 ml) were sequentially added ethyl formate (3.4 ml, 42.2 mmol), and NaH 60% in mineral oil (877 mg, 21.9 mmol), and the reaction was heated in a pre-heated bath at 45° C. until hydrogen evolved (5 minutes). The reaction was then cooled in an ice/water bath and slowly warmed to room temperature overnight. Volatiles were removed under reduced pressure and the residue was triturated with diethyl ether, a precipitate formed and was collected by filtration to provide Intermediate 25-b as a beige solid.

### Step 2: Intermediate 25-c

A solution of Intermediate 25-b (2.0 g, 10.1 mmol) and isobutyrimidamide (756 mg, 8.8 mmol) in dry DMF (17.5 ml) was heated at 100° C. for 1 hour, and then cooled to room temperature. Water and dichloromethane were added, the organic layer was separated, the aqueous phase was extracted twice with dichloromethane, the combined organic extracts were washed with a saturated aqueous solution of ammonium chloride, and brine, dried over MgSO₄, filtered, and concentrated under reduced pressure to provide Intermediate 25-c as a colorless oil.

### Step 3: Intermediate 25-d

To a solution of Intermediate 25-c (1.7 g, 9.4 mmol) in dry THF (37.7 ml) cooled to −15° C. was added dropwise a 1M solution of diisobutyl aluminum hydride in THF (20.7 ml, 20.7 mmol), and the reaction was then stirred for 1 hour. Water (0.8 ml) was slowly added, followed by NaOH 15% (0.85 ml) and water (2.0 ml). The mixture was stirred at room temperature for 30 minutes, MgSO₄ was added, and the mixture was filtered on celite, washed with EtOAc, and the filtrate was reduced under reduced pressure. Purification by silica gel chromatography provided Intermediate 25-d as a colorless oil.

### Step 4: Intermediate 25-e

To a solution of 1-bromo-3-fluoro-5-iodobenzene 2-a (1.5 g, 5.1 mmol) in toluene (2.5 ml) was added Intermediate 25-d (860 mg, 5.6 mmol), 1,10-phenanthroline (185 mg, 1.0 mmol), copper (I) iodide (98 mg, 0.5 mmol), and cesium carbonate (2.3 g, 7.2 mmol). The reaction was stirred at 110° C. for 2 days, and then cooled to room temperature, diluted with ethyl acetate, filtered over celite, and adsorbed over silica gel. Purification by silica gel chromatography provided Intermediate 25-e as a yellow solid.

### Synthesis of Compound 13

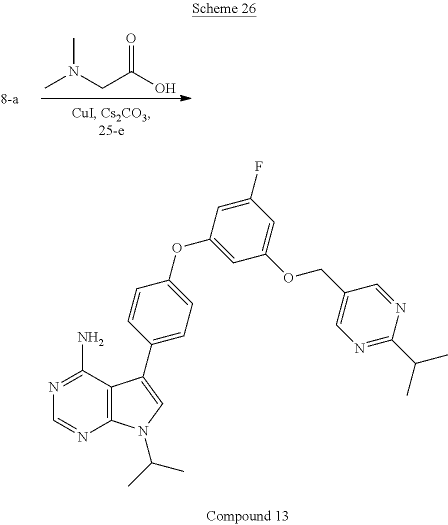

A solution of Intermediate 8-a (90 mg, 0.3 mmol), Intermediate 25-e (109 mg, 0.3 mmol), N,N-Dimethylglycine (104 mg, 1.0 mmol), cesium carbonate (437 mg, 1.3 mmol,) and copper(I) iodide (64 mg, 0.3 mmol) in 1,4-dioxane (0.9 ml) was heated in a pressure vessel, at 110° C. for 2 days, and then cooled to room temperature. Ethyl acetate was added; the reaction was filtered over celite, and adsorbed on silica gel. Purification by silica gel chromatography provided Compound 13 as a white solid. MS (m/z) M+H=513.1.

Compounds 12 and Compound 14 are obtained in a similar manner to Compound 13 starting from commercially available starting materials.

### Synthesis of Intermediate 27-b

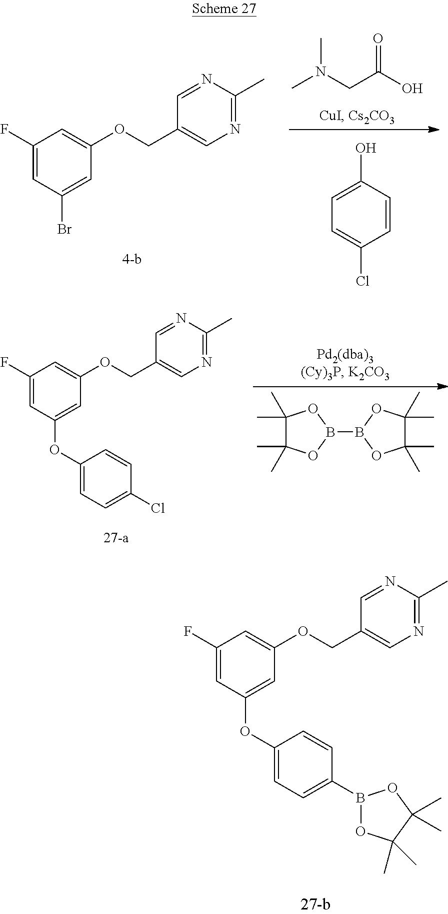

### Step 1: Intermediate 27-a

A solution of Intermediate 4-b (1.5 g, 5.0 mmol), 4-chlorophenol (681 mg, 5.3 mmol), N,N-dimethylglycine (1.5 g, 15.1 mmol), cesium carbonate (8.2 g, 25.2 mmol), and copper (I) iodide (961 mg, 5.0 mmol) in dioxane (14.4 ml) was heated at 110° C. for 2 days, and then cooled to room temperature. Ethyl acetate was added, the reaction was adsorbed on silica gel. Purification by silica gel chromatography provided Intermediate 27-a as a colorless oil.

### Step 2: Intermediate 27-b

To a degassed solution of intermediate 27-a (5.3 g, 15.4 mmol), 4,4,4′,4′,5,5,5′,5′-octamethyl-2,2′-bi(1,3,2-dioxaborolane (4.7 g, 18.4 mmol), potassium acetate (4.5 g, 46.1 mmol), and tricyclohexylphosphine (862 mg, 3.1 mmol) was added Pd₂(dba)₃ (1.4 g, 1.5 mmol) under nitrogen. The reaction was heated in a pressure vessel at 110° C. for 2 days, and then cooled to room temperature. Ethyl acetate was added, the reaction was filtered over celite, and was adsorbed on silica gel. Purification by silica gel chromatography provided Intermediate 27-b as a colorless oil.

### Synthesis of Intermediate 28-d

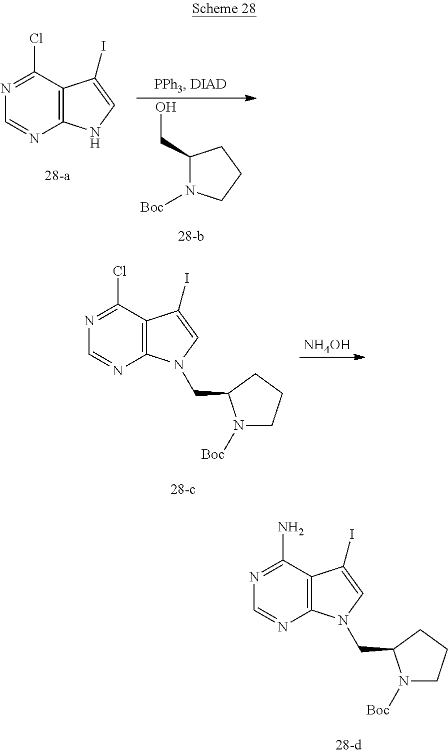

### Step 1: Intermediate 28-c

To a solution of Intermediate 28-a (392 mg, 2.5 mmol) and triphenylphosphine (1.2 g, 4.6 mmol) in THF (27.5 mL) was added Intermediate 28-b (792 mg, 3.9 mmol) and DIAD (904 μl, 4.6 mmol). The solution was stirred at room temperature overnight. Volatiles were removed under reduced pressure. Purification by silica gel chromatography provided Intermediate 28-c as a beige oil.

### Step 2: Intermediate 28-d

To a solution of Intermediate 28-c (1.6 g, 3.5 mmol) in iPrOH (30 ml) was added ammonium hydroxide (40 ml). The reaction mixture was stirred overnight at 90° C., then cooled to room temperature. Volatiles were removed under reduced pressure. The residue was triturated in water; a precipitated formed, and was collected by filtration to provide Intermediate 28-d as a beige solid.

### Synthesis of Compound 10

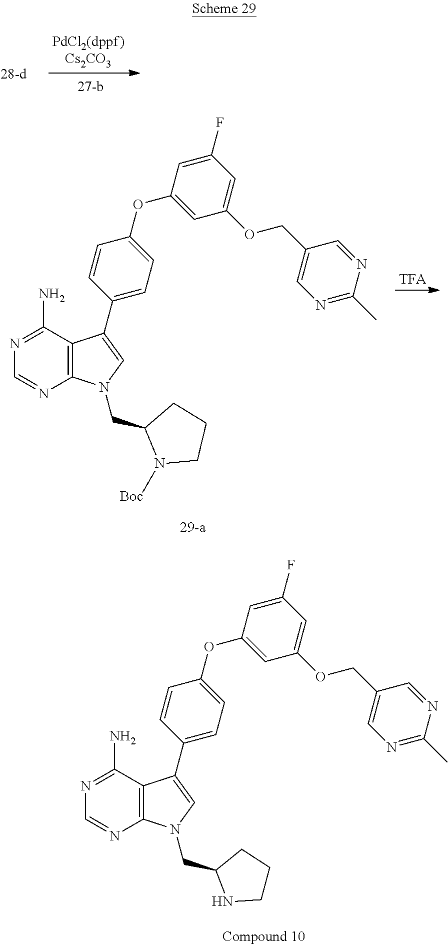 [SMILES: Cc1ncc(COc2cc(F)cc(Oc3ccc(-c4cn(C[C@H]5CCCN5C(=O)OC(C)(C)C)c5ncnc(N)c45)cc3)c2)cn1 ; Cc1ncc(COc2cc(F)cc(Oc3ccc(-c4cn(C[C@H]5CCCN5)c5ncnc(N)c45)cc3)c2)cn1 ; "TFA"]

### Step 1: Intermediate 29-a

To a degassed solution of Intermediate 28-d (300 mg, 0.7 mmol), Intermediate 27-b (354 mg, 0.8 mmol), and cesium carbonate (662 mg, 2.0 mmol) in DME (3.6 ml), and water (0.9 ml) was added PdCl₂(dppf) (50 mg, 0.07 mmol), and the reaction was heated in a pressure vessel at 100° C. overnight, and then cooled to room temperature. Ethyl acetate was added, the reaction was adsorbed on silica gel. Purification by silica gel chromatography provided Intermediate 29-a as a white solid.

### Step 2: Compound 10

A solution of Intermediate 29-a (159 mg, 0.2 mmol) in TFA (3 ml) was stirred for 15 minutes. Volatiles were removed under reduced pressure to provide Compound 10.2TFA as a white solid.

Compound 8 is obtained in a similar manner to Compound 10 starting from commercially available starting materials.

### Synthesis of Compound 22

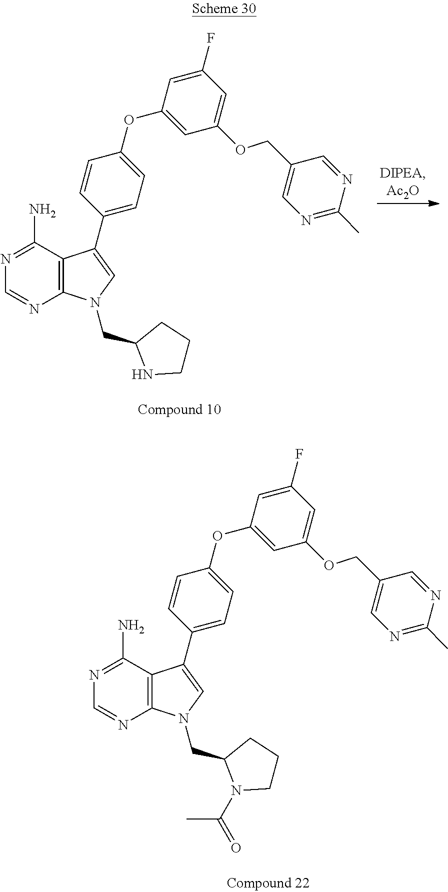 [SMILES: Cc1ncc(COc2cc(F)cc(Oc3ccc(-c4cn(C[C@H]5CCCN5)c5ncnc(N)c45)cc3)c2)cn1 ; "DIPEA,Ac2O" ; CC(=O)N1CCC[C@@H]1Cn1cc(-c2ccc(Oc3cc(F)cc(OCc4cnc(C)nc4)c3)cc2)c2c(N)ncnc21]

To a solution of Compound 10 (170 mg, 0.3 mmol) in dichloromethane (3 ml) was added DIPEA (282 μl, 1.6 mmol), and acetic anhydride (356 μl, 0.3 mmol), and the reaction was stirred at room temperature overnight. Volatiles were removed under reduced pressure. Purification by silica gel chromatography provided Compound 22 as a white solid. MS (m/z) M+H=568.1.

### Synthesis of Intermediate 31-h

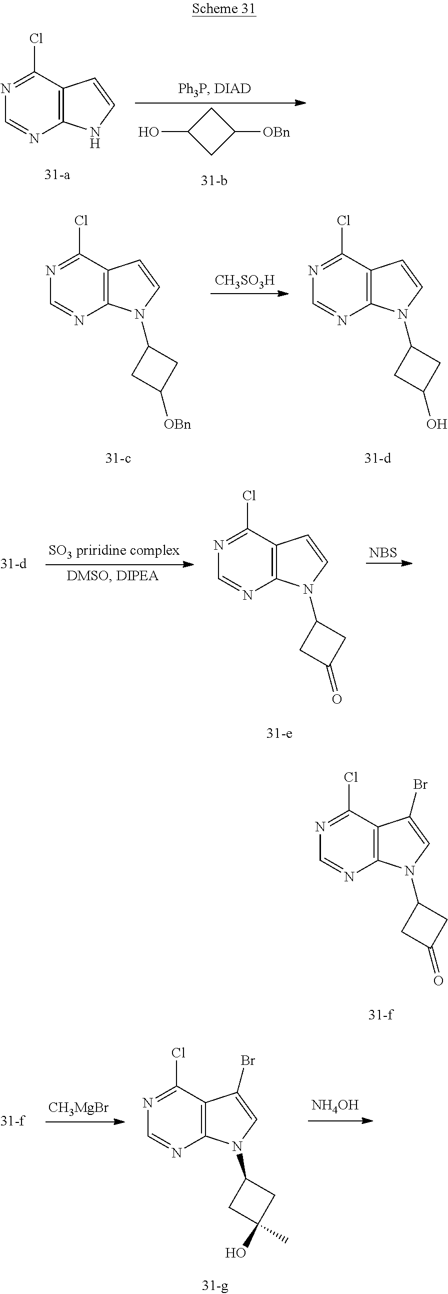

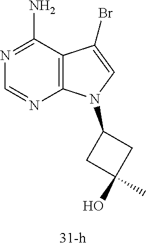 [SMILES: C[C@]1(O)C[C@@H](n2cc(Br)c3c(N)ncnc32)C1]

### Step 1: Intermediate 31-c

To a solution of 4-chloro-7H-pyrrolo[2,3-d]pyrimidine 31-a (392 mg, 2.5 mmol) and Intermediate 31-b (500 mg, 2.8 mmol) in THF (12.7 mL) were sequentially added triphenylphosphine (2.0 g, 7.6 mmol), and DIAD (1.5 ml, 7.6 mmol). The solution was stirred at room temperature for 1 hour. Volatiles were removed under reduced pressure. Purification by silica gel chromatography provided Intermediate 5-c as a beige oil.

### Step 2: Intermediate 31-d

A solution of Intermediate 5-c (680 mg, 2.2 mmol) in methane sulfonic acid (7 ml) and chloroform (14 ml) was stirred at room temperature overnight. A saturated aqueous solution of NaHCO₃, and ethyl acetate were added, the organic layer was separated, washed with brine, dried over MgSO₄, filtered, and concentrated under reduced pressure to provide Intermediate 5-d as a beige oil.

### Step 3: Intermediate 31-e

To a solution of Intermediate 31-d (500 mg, 2.2 mmol) in dichloromethane (22.3 ml) cooled to 0° C. were sequentially added DIPEA (1.6 ml, 8.9 mmol), and SO₃ pyridine complex (1.0 g, 6.7 mmol) in DMSO (3.0 ml), and the reaction was then stirred overnight at 0° C. Water and ethyl acetate were added; the organic layer was separated, washed with 1N HCl, a saturated aqueous solution of NaHCO₃, and brine, dried over MgSO₄, filtered, and concentrated under reduced pressure to provide Intermediate 31-e as a beige oil.

### Step 4: Intermediate 31-f

To a solution of Intermediate 31-e (500 mg, 2.2 mmol) in DMF (5.6 ml) cooled to 0° C., was slowly added a 0.7N solution of N-bromosuccinimide in DMF (3.5 ml, 2.5 mmol). The reaction mixture was stirred for 15 minutes at 0° C. Water was added; a precipitate formed and was collected by filtration. Purification by silica gel chromatography provided Intermediate 31-f as a beige solid.

### Step 5: Intermediate 31-g

To a solution of Intermediate 31-f (260 mg, 0.8 mmol) in THF (2.1 ml) cooled to −78° C. then was slowly added a 1M solution of methylmagnesium bromide in THF (1.7 ml, 1.7 mmol) under nirogen. The reaction mixture was stirred for 2 hours at −78° C., quenched by slow addition of a saturated aqueous solution of ammonium chloride, and warmed to room temperature. Ethyl acetate was added, the organic layer was separated, the aqueous phase was extracted twice with ethyl acetate, the combined organic extracts were washed with brine, dried over anhydrous MgSO₄, filtered, and concentrated under reduced pressure to provide Intermediate 31-g as a white solid.

### Step 6: Intermediate 5-h

To a solution of Intermediate 31-g (260 mg, 0.8 mmol) in iPrOH (2.0 ml) was added ammonium hydroxide (2.0 ml). The reaction mixture was stirred overnight at 90° C., and then cooled to room temperature. Volatiles were removed under reduced pressure. The residue was triturated in water; a precipitated formed, and was collected by filtration to provide Intermediate 31-h as a beige solid.

### Synthesis of Compound 18

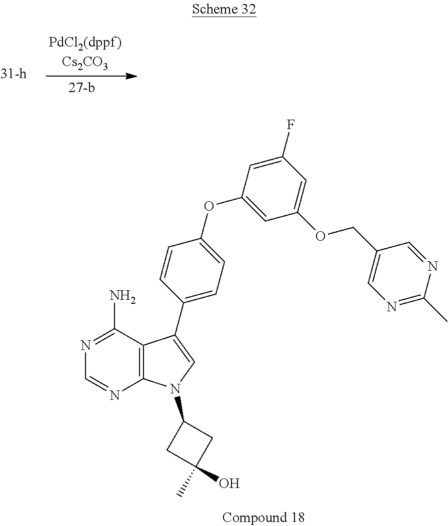 [SMILES: Cc1ncc(COc2cc(F)cc(Oc3ccc(-c4cn([C@H]5C[C@@](C)(O)C5)c5ncnc(N)c45)cc3)c2)cn1]

To a degassed solution of Intermediate 31-h (234 mg, 0.8 mmol), Intermediate 27-b (412 mg, 0.9) mmol), and cesium carbonate (770 mg, 2.4 mmol) in DME (4.2 ml), and water (1.0 ml) was added PdCl₂(dppf) (58 mg, 0.08 mmol), and the reaction was heated in a pressure vessel at 100° C. overnight, and then cooled to room temperature. Ethyl acetate was added, the reaction was adsorbed on silica gel. Purification by reverse phase chromatography eluting with a 0.1% formic acid/methanol gradient provided Compound 18 as a white solid. MS (m/z) M+H=527.1.

### Synthesis of Intermediate 33-d

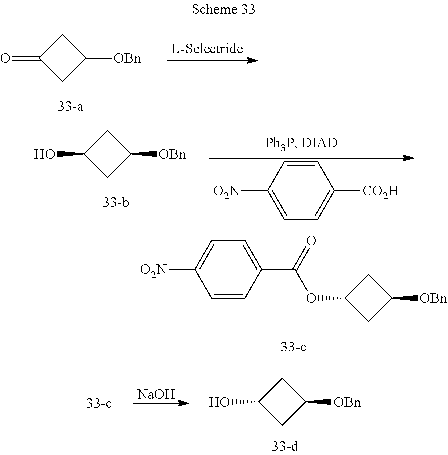

### Step 1: Intermediate 33-b

To a solution of 3-(benzyloxy)cyclobutanone 33-a (5.0 g, 28.4 mmol) in THF (28.4 ml) cooled to −78° C. was added a 1.0 M solution L-Selectride in THF (31.2 ml, 31.2 mmol), and the reaction was stirred at −78° C. for 1 hour, and then at room temperature for 1 hour. A saturated aqueous solution of NaHCO₃ was slowly added. The mixture was cooled to 0° C. and 30% aqueous H₂O₂ (4 ml)) was added dropwise. Water and ethyl acetate were added, the organic layer was separated, washed with brine, dried over MgSO₄, filtered, and concentrated under reduced pressure to provide Intermediate 33-b as a colorless oil.

### Step 2: Intermediate 33-c

To a solution of Intermediate 33-b (5.0 g, 28.4 mmol), 4-nitrobenzoic acid (7.1 g, 42.6 mmol), and triphenylphosphine (11.2 g, 42.6 mmol) in THF (71.0 ml) cooled to 0° C. was added DIAD (8.3 ml, 42.6 mmol) dropwise. The reaction was then stirred at room temperature overnight. Volatiles were removed under reduced pressure. Purification by silica gel chromatography provided Intermediate 33-c as a yellow solid.

### Step 3: Intermediate 33-d

To a solution of Intermediate 33-c (3.9 g, 11.8 mmol) in 1,4-dioxane (13.1 ml) was added a 2 M aqueous solution of sodium hydroxide (23.6 ml, 47.2 mmol), and the reaction was stirred at room temperature overnight. Ethyl acetate was added, the organic layer was separated, washed with brine, dried over MgSO₄, filtered, and concentrated under reduced pressure to provide Intermediate 33-d as a yellow oil.

### Synthesis of Intermediate 34-d

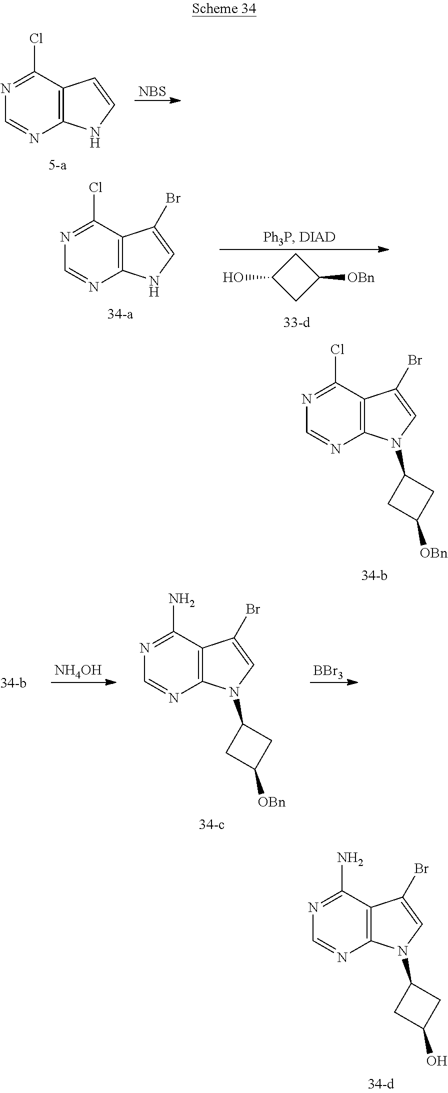

### Step 1: Intermediate 34-a

To a solution of 4-chloro-7H-pyrrolo[2,3-d]pyrimidine 5-a (5.0 g, 32.6 mmol) in DMF (81.0 ml) cooled to 0° C. was added N-bromosuccinimide (6.4 g, 35.8 mmol) in small portions. After the addition was competed, the reaction was stirred at room temperature for 15 minutes. Water was added, a precipitate formed, and was collected by filtration to provide Intermediate 34-a as a white solid.

### Step 2: Intermediate 34-b

To a solution of Intermediate 34-a (2.9 g, 12.7 mmol), Intermediate 33-d (2.5 g, 14.0 mmol), and triphenylphosphine (5.0 g, 19.1 mmol) in THF (32.0 ml) cooled to 0° C. was added DIAD (3.7 ml, 19.1 mmol) dropwise. After the addition was completed, the reaction was stirred at room temperature for 3 days. Volatiles were removed under reduced pressure. Purification by silica gel chromatography provided Intermediate 34-b as a white solid.

### Step 3: Intermediate 34-c

To a solution of Intermediate 34-b (3.3 g, 8.5 mmol) in iPrOH (2.0 ml) was added ammonium hydroxide (3.3 ml). The reaction mixture was stirred overnight at 90° C. in a pressure vessel, and then cooled to room temperature. Water and ethyl acetate were added; the organic layer was separated, washed with a saturated aqueous solution of NaHCO₃ and brine, dried over MgSO₄, filtered, and concentrated under reduced pressure to provide Intermediate 34-c as a yellow solid.

### Step 4: Intermediate 34-d

To a solution of Intermediate 34-c (3.1 g, 8.4 mmol) in dichloromethane (84 ml) cooled to −78° C. was added a 1M solution of boron in dicloromethane (12.6 ml, 12.6 mmol), and the reaction was stirred at −78° C. for 30 minutes, and then at room temperature until completion. A saturated aqueous solution of NaHCO₃ was slowly added, a precipitate formed, and was collected by filtration, washed water, and dried in vacuo to provide Intermediate 34-d as a white solid.

### Synthesis of Compound 26

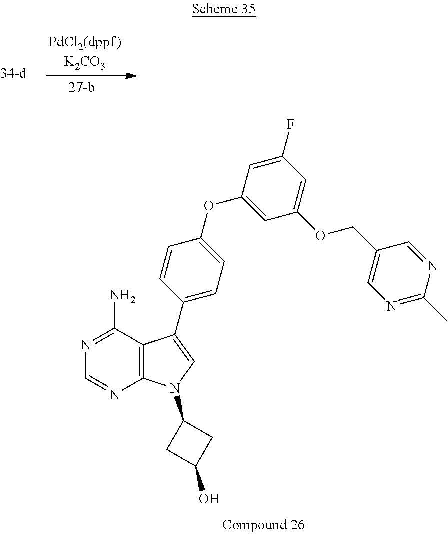 [SMILES: Cc1ncc(COc2cc(F)cc(Oc3ccc(-c4cn([C@H]5C[C@@H](O)C5)c5ncnc(N)c45)cc3)c2)cn1]

To a degassed solution of Intermediate 34-d (500 mg, 1.8 mmol), Intermediate 27-b (925 mg, 2.1 mmol), and potassium carbonate (732 mg, 5.3 mmol) in DME (9.4 ml), and water (2.3 ml) was added PdCl₂(dppf) (129 mg, 0.2 mmol), and the reaction was heated in a pressure vessel at 100° C. for 2 hours, and then cooled to room temperature. A saturated aqueous solution of ammonium chloride and ethyl acetate were added, the organic layer was separated, washed with brine, dried over MgSO₄, filtered, and concentrated under reduced pressure. Purification by reverse phase chromatography eluting with a 0.1% formic acid/methanol gradient provided Compound 26 as a white solid. MS (m/z) M+H=513.2.

### Synthesis of Intermediate 36-b

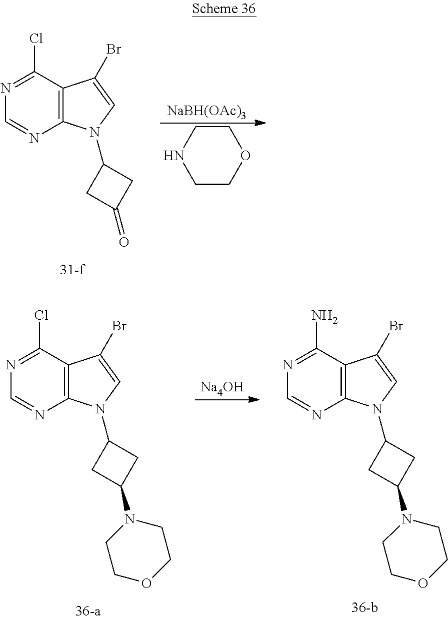

### Step 1: Intermediate 36-a

To a solution of Intermediate 31-f (760 mg, 2.5 mmol) in THF (25.3 ml) cooled to 0° C. was added morpholine (218 μl, 2.5 mmol) and sodium triacethoxyborohydride (1.2 g, 5.7 mmol) and the reaction was slowly warmed to room temperature and stirred overnight. Volatiles were removed under reduced pressure. A saturated aqueous solution of NaHCO₃ and dichloromethane were added, the organic layer was separated, washed with brine, dried over MgSO₄, filtered, and concentrated under reduced pressure. Purification by silica gel chromatography provided Intermediate 36-a as a white solid.

### Step 2: Intermediate 36-b

To a solution of Intermediate 36-a (940 mg, 2.5 mmol) in iPrOH (3.5 ml) was added ammonium hydroxide (4.9 ml). The reaction mixture was stirred overnight at 90° C. in a pressure vessel and then cooled to room temperature. Volatiles were removed under reduced pressure. Purification by silica gel chromatography provided Intermediate 36-b as a white solid.

### Synthesis of Compound 23

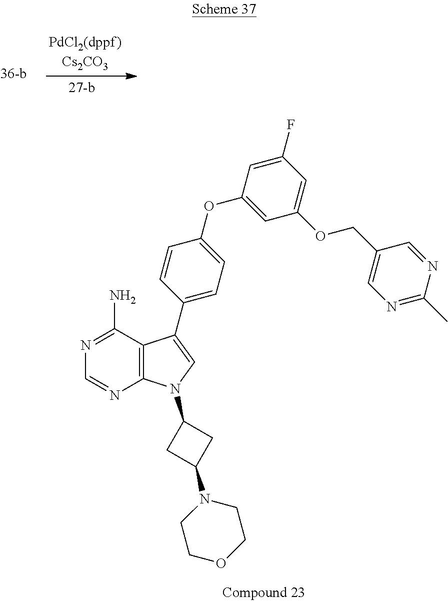 [SMILES: Cc1ncc(COc2cc(F)cc(Oc3ccc(-c4cn([C@H]5C[C@@H](N6CCOCC6)C5)c5ncnc(N)c45)cc3)c2)cn1]

To a degassed solution of Intermediate 36-b (75 mg, 0.2 mmol), Intermediate 27-b (102 mg, 0.2 mmol) and cesium carbonate (208 mg, 0.6 mmol) in DME (1.1 ml), and water (0.3 ml) was added PdCl₂(dppf) (16 mg, 0.02 mmol), and the reaction was heated in a pressure vessel at 100° C. overnight, and then cooled to room temperature. Ethyl acetate was added and the reaction was adsorbed on silica gel. Purification by reverse phase chromatography eluting with a 0.1% formic acid/methanol gradient provided Compound 23 as a beige solid. MS (m/z) M+H=582.2.

### Synthesis of Intermediate 38-c

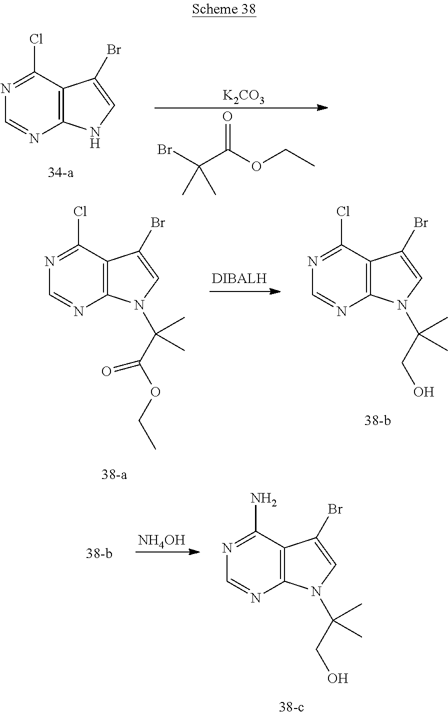

### Step 1: Intermediate 38-a

To a solution of Intermediate 34-a (250 mg, 1.1 mmol), and potassium carbonate (297 mg, 2.1 mmol) in DMF (5.4 ml) was stirred at room temperature for 2 weeks. A saturated aqueous solution of ammonium chloride and ethyl acetate were added, the organic layer was separated, washed with brine, dried over MgSO₄, filtered, and concentrated under reduced pressure. Purification by silica gel chromatography provided Intermediate 38-a as a white solid

### Step 2: Intermediate 38-b

To a solution of Intermediate 38-a (223 mg, 0.6 mmol) in THF (1.3 ml) cooled to 0° C., were added DIBAL-H (2.6 ml, 2.6 mmol), and the reaction was stirred overnight. 100 μl of water and 100 μl of 15% aqueous NaOH were slowly added. After stirring for 5 minutes, 260 μl of water were added. The mixture was stirred at room temperature for 30 minutes, MgSO₄ was added, and the mixture was filtered on celite, washed with EtOAc, and the filtrate was reduced under reduced pressure. Purification by silica gel chromatography provided Intermediate 38-b as a colorless oil.

### Step 3: Intermediate 38-c

To a solution of Intermediate 38-b (190 mg, 0.6 mmol) in iPrOH (1.6 ml) was added ammonium hydroxide (1.6 ml). The reaction mixture was stirred overnight at 90° C. in a pressure vessel, and then cooled to room temperature. Volatiles were removed under reduced pressure. Water and ethyl acetate were added, the organic layer was separated, washed with brine, dried over MgSO₄, filtered, and concentrated under reduced pressure to provide Intermediate 38-c as a white solid.

### Synthesis of Compound 24

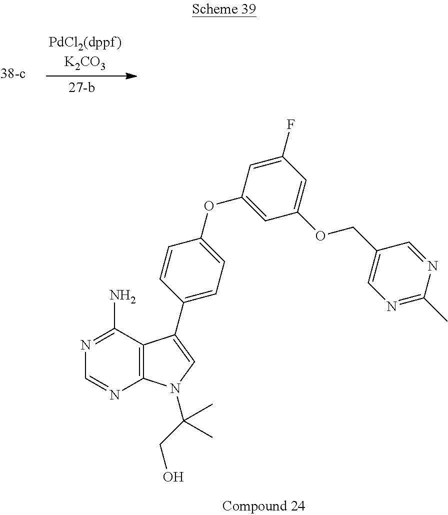 [SMILES: Cc1ncc(COc2cc(F)cc(Oc3ccc(-c4cn(C(C)(C)CO)c5ncnc(N)c45)cc3)c2)cn1]

To a degassed solution of Intermediate 38-c (150 mg, 0.5 mmol), Intermediate 27-b (321 mg, 0.7 mmol), and potassium carbonate (218 mg, 1.6 mmol) in DME (2.8 ml), and water (0.7 ml) was added PdCl₂(dppf) (38 mg, 0.05 mmol), and the reaction was heated in a pressure vessel at 100° C. overnight, and then cooled to room temperature. A saturated aqueous solution of ammonium chloride and ethyl acetate were added, the organic layer was separated, washed with brine, dried over MgSO₄, filtered, and concentrated under reduced pressure. Purification by reverse phase chromatography eluting with a 0.1% formic acid/methanol gradient provided Compound 24 as a beige solid. MS (m/z) M+H=515.2.

### Synthesis of Intermediate 40-d

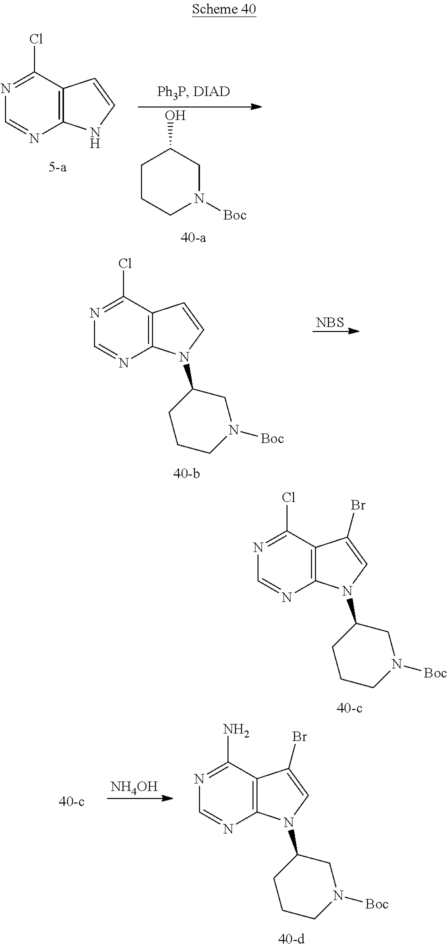

### Step 1: Intermediate 40-b

To a solution of 4-chloro-7H-pyrrolo[2,3-d]pyrimidine 5-a (2.0 g, 13.0 mmol), (S)-tert-butyl 3-hydroxypiperidine-1-carboxylate 40-a (5.2 g, 26.0 mmol), and polymer supported triphenylphosphine (3 mmol/g) (26.0 mmol) in THF (52.1 ml) was added DIAD (5.0 ml, 26.0 mmol), and the reaction was stirred at room temperature for 3 days, and then filtered. The filtrate was reduced under reduced pressure. Purification by silica gel chromatography provided Intermediate 40-b as a beige foam.

### Step 2: Intermediate 40-c

To a solution of Intermediate 40-b (3.5 g, 10.4 mmol) in DMF (26.0 ml) cooled to 0° C. was added N-bromosuccinimide (2.0 g, 11.4 mmol) in small portions. After the addition was competed, the reaction was stirred at room temperature for 15 minutes. Water and ethyl acetate were added, the organic layer was separated, washed with a saturated aqueous solution of ammonium chloride and brine, dried over MgSO₄, filtered, and concentrated under reduced pressure to provide Intermediate 40-c as a beige foam.

### Step 3: Intermediate 40-d

To a solution of Intermediate 40-c (3.5 g, 8.4 mmol) in dioxane (21.0 ml) was added ammonium hydroxide (21.0 ml). The reaction mixture was stirred overnight at 90° C. in a pressure vessel and then cooled to room temperature. Volatiles were removed under reduced pressure. Water and ethyl acetate were added, the organic layer was separated, washed with brine, dried over MgSO₄, filtered, and concentrated under reduced pressure. Purification by silica gel chromatography provided Intermediate 40-d as a white solid.

### Synthesis of Compound 27

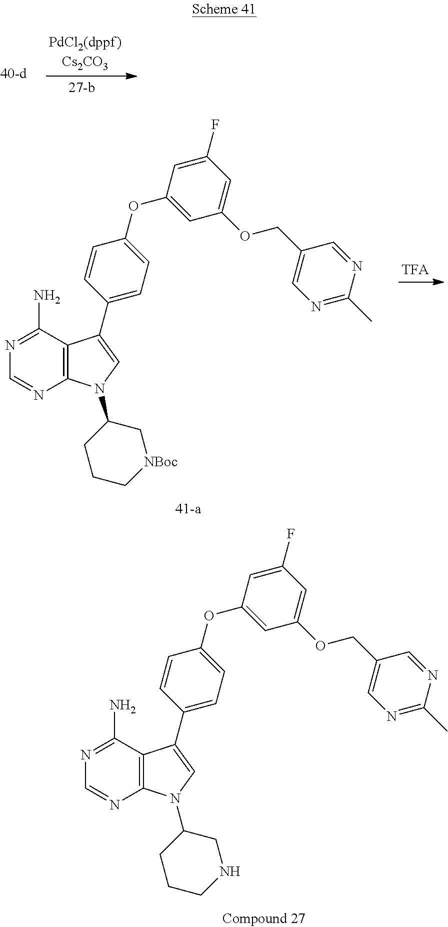 [SMILES: Cc1ncc(COc2cc(F)cc(Oc3ccc(-c4cn([C@@H]5CCCN(C(=O)OC(C)(C)C)C5)c5ncnc(N)c45)cc3)c2)cn1 ; Cc1ncc(COc2cc(F)cc(Oc3ccc(-c4cn(C5CCCNC5)c5ncnc(N)c45)cc3)c2)cn1]

### Step 1: Intermediate 41-a

To a degassed solution of Intermediate 40-d (250 mg, 0.6 mmol), Intermediate 27-b (385 mg, 0.9 mmol), and potassium carbonate (262 mg, 1.9 mmol) in DME (3.4 ml), and water (0.8 ml) was added PdCl₂(dppf) (46 mg, 0.06 mmol), and the reaction was heated in a pressure vessel at 100° C. overnight, and then cooled to room temperature. A saturated aqueous solution of ammonium chloride and ethyl acetate were added, the organic layer was separated, washed with brine, dried over MgSO₄, filtered, and concentrated under reduced pressure. Purification by silica gel chromatography provided Intermediate 41-a as a beige foam.

### Step 2: Compound 27

A solution of intermediate 41-a (100 mg, 0.2 mmol) in methanol (0.5 ml) was added a 4 N solution of HCl in 1,4-dioxane (1 ml) and the reaction was stirred at room temperature until completion. Volatiles were removed under reduced pressure to provide Compound 27.3HCl as a white solid. MS (m/z) M+H=526.2.

### Synthesis of Compound 25

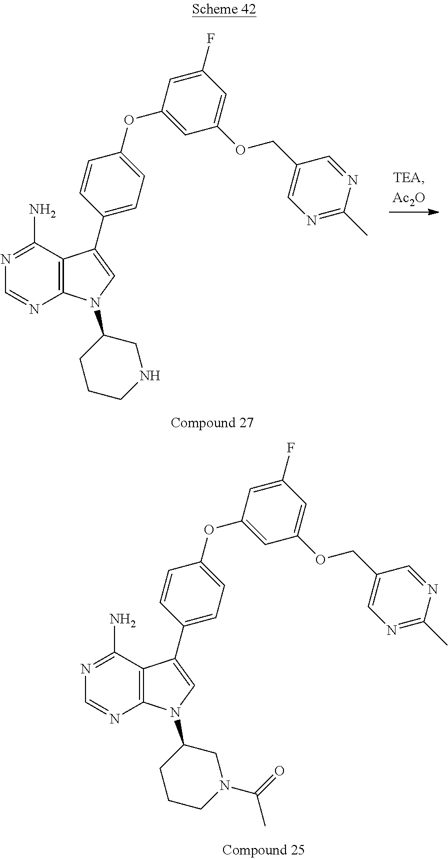 [SMILES: Cc1ncc(COc2cc(F)cc(Oc3ccc(-c4cn([C@@H]5CCCNC5)c5ncnc(N)c45)cc3)c2)cn1 ; CC(=O)N1CCC[C@@H](n2cc(-c3ccc(Oc4cc(F)cc(OCc5cnc(C)nc5)c4)cc3)c3c(N)ncnc32)C1]

To a solution of Compound 27 (100 mg, 0.1 mmol) in dichloromethane (1.5 ml) cooled to 0° C. were sequentially added triethylamine (87 μl, 0.6 mmol), and a 1.0 M solution of acetic anhydride (156 μl, 0.15 mmol), and the reaction was stirred at 0° C. for 1 hour. Volatiles were removed under reduced pressure. Purification by silica gel chromatography provided Compound 25 as a white solid. MS (m/z) M+H=568.2.

TABLE 1
Example Compounds of Formula 1

| Compound | Structure | MS (m/z) |
|---|---|---|
| 1 | 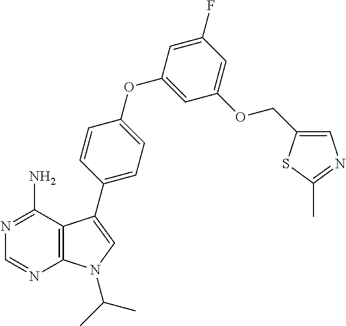 [SMILES: Cc1ncc(COc2cc(F)cc(Oc3ccc(-c4cn(C(C)C)c5ncnc(N)c45)cc3)c2)s1] | [M + H]⁺ = 490.2; |
| 2 | 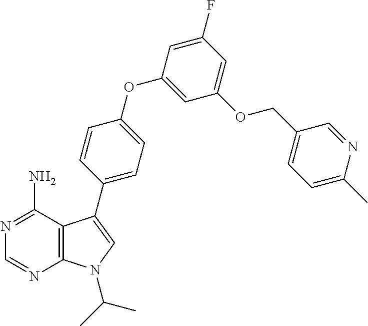 [SMILES: Cc1ccc(COc2cc(F)cc(Oc3ccc(-c4cn(C(C)C)c5ncnc(N)c45)cc3)c2)cn1] | [M + H]⁺ = 484.2; |
| 3 | 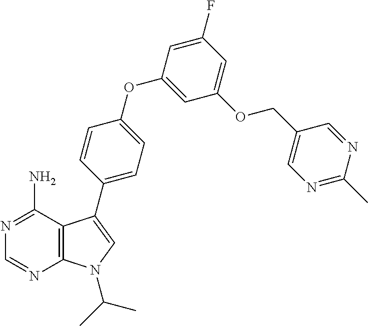 [SMILES: Cc1ncc(COc2cc(F)cc(Oc3ccc(-c4cn(C(C)C)c5ncnc(N)c45)cc3)c2)cn1] | [M + H]⁺ = 485.2; |
| 4 | 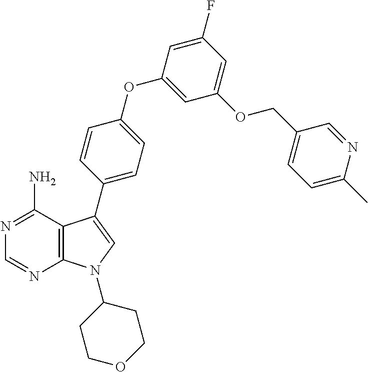 [SMILES: Cc1ccc(COc2cc(F)cc(Oc3ccc(-c4cn(C5CCOCC5)c5ncnc(N)c45)cc3)c2)cn1] | [M + H]⁺ = 526.3; |
| 5 | 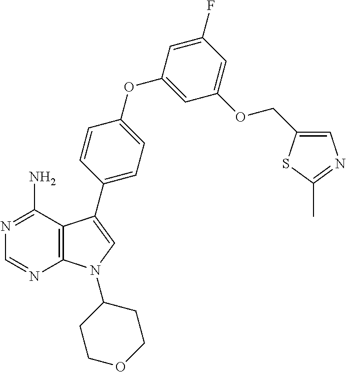 [SMILES: Cc1ncc(COc2cc(F)cc(Oc3ccc(-c4cn(C5CCOCC5)c5ncnc(N)c45)cc3)c2)s1] | [M + H]⁺ = 532.3; |
| 6 | 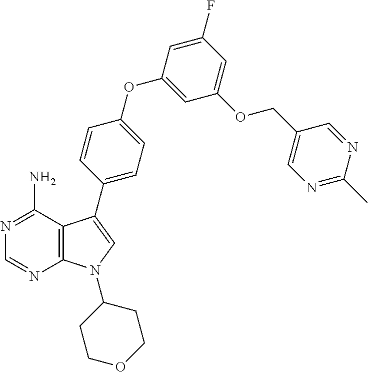 [SMILES: Cc1ncc(COc2cc(F)cc(Oc3ccc(-c4cn(C5CCOCC5)c5ncnc(N)c45)cc3)c2)cn1] | [M + H]⁺ = 527.2; |
| 7 | 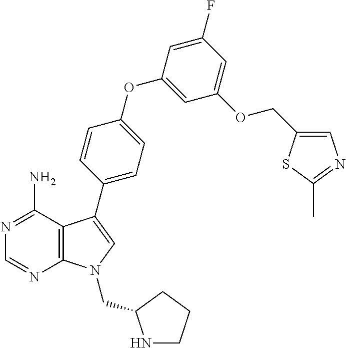 [SMILES: Cc1ncc(COc2cc(F)cc(Oc3ccc(-c4cn(C[C@@H]5CCCN5)c5ncnc(N)c45)cc3)c2)s1] | [M + H]⁺ = 531.2; |
| 8 | 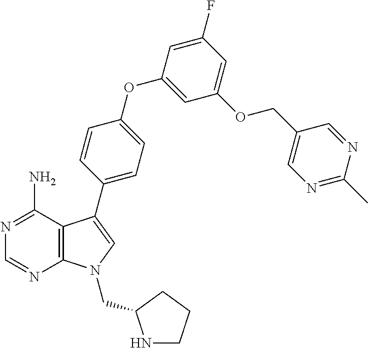 [SMILES: Cc1ncc(COc2cc(F)cc(Oc3ccc(-c4cn(C[C@@H]5CCCN5)c5ncnc(N)c45)cc3)c2)cn1] | [M + H]⁺ = 526.2; |
| 9 | 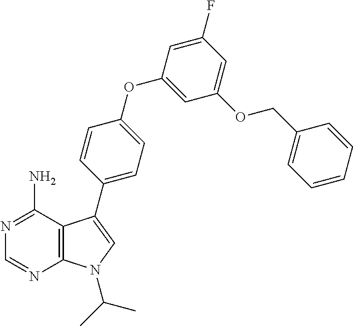 [SMILES: CC(C)n1cc(-c2ccc(Oc3cc(F)cc(OCc4ccccc4)c3)cc2)c2c(N)ncnc21] | [M + H]⁺ = 469.0; |
| 10 | 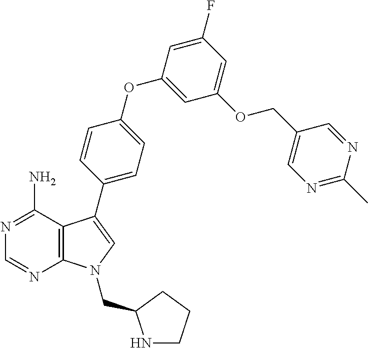 [SMILES: Cc1ncc(COc2cc(F)cc(Oc3ccc(-c4cn(C[C@H]5CCCN5)c5ncnc(N)c45)cc3)c2)cn1] | [M + H]⁺ = 526.1; |
| 11 | 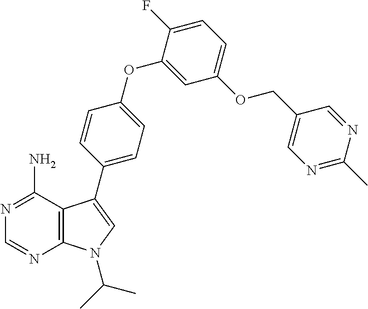 [SMILES: Cc1ncc(COc2ccc(F)c(Oc3ccc(-c4cn(C(C)C)c5ncnc(N)c45)cc3)c2)cn1] | [M + H]⁺ = 485.1; |
| 12 | 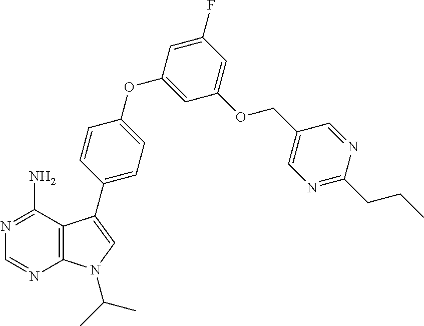 [SMILES: CCCc1ncc(COc2cc(F)cc(Oc3ccc(-c4cn(C(C)C)c5ncnc(N)c45)cc3)c2)cn1] | [M + H]⁺ = 513.1; |
| 13 | 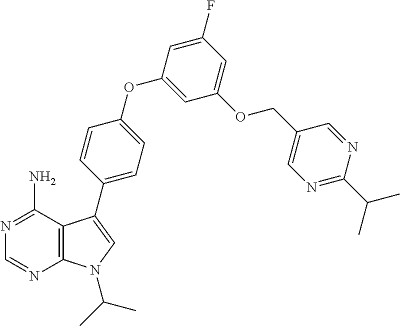 [SMILES: CC(C)c1ncc(COc2cc(F)cc(Oc3ccc(-c4cn(C(C)C)c5ncnc(N)c45)cc3)c2)cn1] | [M + H]⁺ = 513.1; |
| 14 | 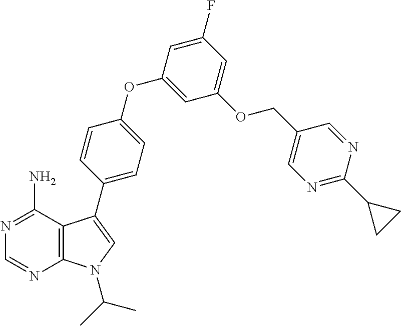 [SMILES: CC(C)n1cc(-c2ccc(Oc3cc(F)cc(OCc4cnc(C5CC5)nc4)c3)cc2)c2c(N)ncnc21] | [M + H]⁺ = 511.1; |
| 15 | 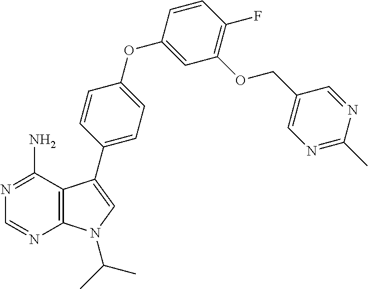 [SMILES: Cc1ncc(COc2cc(Oc3ccc(-c4cn(C(C)C)c5ncnc(N)c45)cc3)ccc2F)cn1] | [M + H]⁺ = 485.1; |
| 16 | 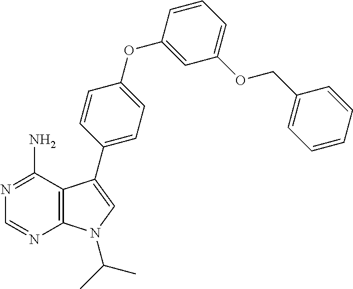 [SMILES: CC(C)n1cc(-c2ccc(Oc3cccc(OCc4ccccc4)c3)cc2)c2c(N)ncnc21] | [M + H]⁺ = 451.2; |
| 17 | 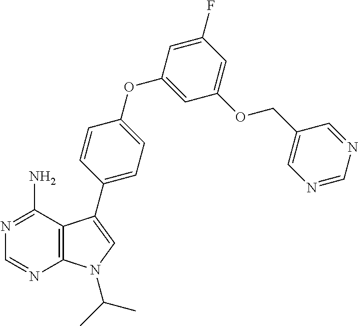 [SMILES: CC(C)n1cc(-c2ccc(Oc3cc(F)cc(OCc4cncnc4)c3)cc2)c2c(N)ncnc21] | [M + H]⁺ = 471.1; |
| 18 | 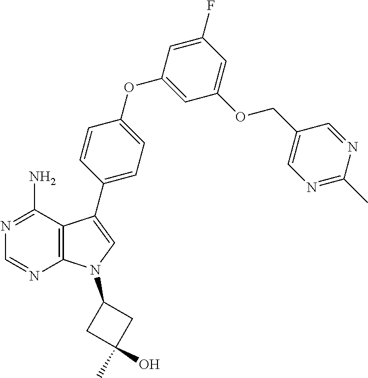 [SMILES: Cc1ncc(COc2cc(F)cc(Oc3ccc(-c4cn([C@H]5C[C@@](C)(O)C5)c5ncnc(N)c45)cc3)c2)cn1] | [M + H]⁺ = 527.1; |
| 19 | 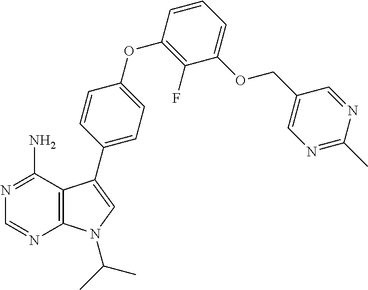 [SMILES: Cc1ncc(COc2cccc(Oc3ccc(-c4cn(C(C)C)c5ncnc(N)c45)cc3)c2F)cn1] | [M + H]⁺ = 485.1; |
| 20 | 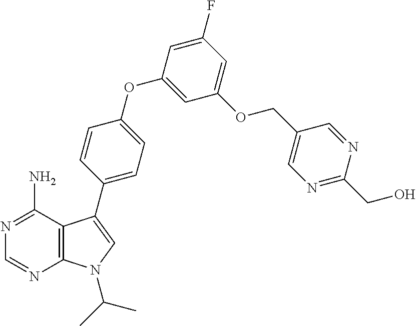 [SMILES: CC(C)n1cc(-c2ccc(Oc3cc(F)cc(OCc4cnc(CO)nc4)c3)cc2)c2c(N)ncnc21] | [M + H]⁺ = 501.1; |
| 21 | 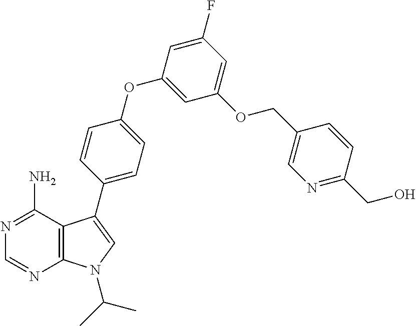 [SMILES: CC(C)n1cc(-c2ccc(Oc3cc(F)cc(OCc4ccc(CO)nc4)c3)cc2)c2c(N)ncnc21] | [M + H]⁺ = 500.2; |
| 22 | 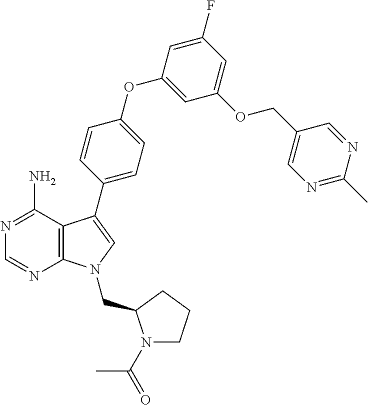 [SMILES: CC(=O)N1CCC[C@@H]1Cn1cc(-c2ccc(Oc3cc(F)cc(OCc4cnc(C)nc4)c3)cc2)c2c(N)ncnc21] | [M + H]⁺ = 568.1; |
| 23 | 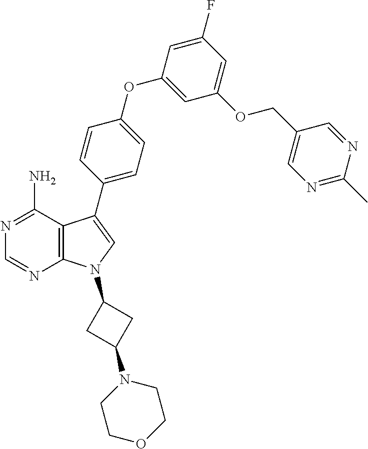 [SMILES: Cc1ncc(COc2cc(F)cc(Oc3ccc(-c4cn([C@H]5C[C@@H](N6CCOCC6)C5)c5ncnc(N)c45)cc3)c2)cn1] | [M + H]⁺ = 582.2; |
| 24 | 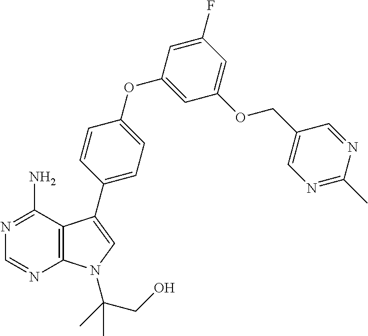 [SMILES: Cc1ncc(COc2cc(F)cc(Oc3ccc(-c4cn(C(C)(C)CO)c5ncnc(N)c45)cc3)c2)cn1] | [M + H]⁺ = 515.2; |
| 25 | 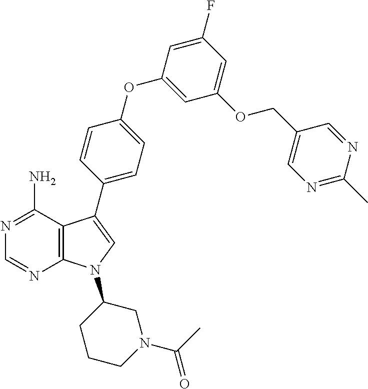 [SMILES: CC(=O)N1CCC[C@@H](n2cc(-c3ccc(Oc4cc(F)cc(OCc5cnc(C)nc5)c4)cc3)c3c(N)ncnc32)C1] | [M + H]⁺ = 568.2; |
| 26 | 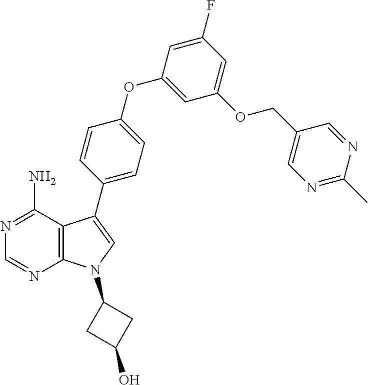 [SMILES: Cc1ncc(COc2cc(F)cc(Oc3ccc(-c4cn([C@H]5C[C@@H](O)C5)c5ncnc(N)c45)cc3)c2)cn1] | [M + H]⁺ = 513.2, or |
| 27 | 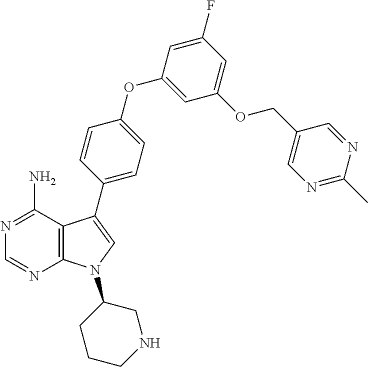 [SMILES: Cc1ncc(COc2cc(F)cc(Oc3ccc(-c4cn([C@@H]5CCCNC5)c5ncnc(N)c45)cc3)c2)cn1] | [M + H]⁺ = 526.2 |

Biological Assays

Assays for determining kinase activity are described in more detail in the accompanying examples.

Kinase Inhibition

Btk Kinase Inhibition Assays

Method A

Fluorescence polarization-based kinase assays were performed in 384 well-plate format using histidine tagged recombinant human full-length Bruton Agammaglobulinemia Tyrosine Kinase (Btk) and a modified protocol of the KinEASE™ FP Fluorescein Green Assay supplied from Millipore®. Kinase reaction were performed at room temperature for 60 minutes in presence of 250 μM substrate, 10 μM ATP and variable test article concentrations. The reaction was stopped with EDTA/kinease detection reagents. Phosphorylation of the substrate peptide was detected by fluorescence polarization measured with a Tecan 500 instrument. From the dose-response curve obtained, the IC₅₀ was calculated using Graph Pad Prisms® using a non linear fit curve. The Km for ATP on each enzyme was experimentally determined and the Ki values calculated using the Cheng-Prusoff equation (see: Cheng Y, Prusoff W. H. (1973) Relationship between the inhibition constant (K1) and the concentration of inhibitor which causes 50% inhibition (I₅₀) of an enzymatic reaction”. Biochem Pharmacol 22 (23): 3099-108). kᵢ values are reported in Tables 2a and 2b:

TABLE 2a
Inhibition of Btk

| Compound | EC₅₀ (nM) |
|---|---|
| 1 | a |
| 2 | a |
| 3 | a |
| 4 | a |
| 5 | a |
| 6 | a |
| 7 | a |
| 8 | a |
| 9 | a |
| 10 | b |
| 11 | a |
| 12 | a |
| 13 | a |
| 14 | a |
| 15 | b |
| 17 | a |
| 18 | a |
| 23 | a |

a - Ki <100 nM;
b - 100 nM < Ki < 1000 nM,
c - ki >1000 nM

Method B

In vitro potency of selected compound was defined against human BTK kinase (hBTK) using KinaseProfiler radiometric protein kinase assays performed at Eurofins Pharma Discovery Services UK Limited.

hBTK kinase is diluted in buffer and all compounds were prepared to 50× final assay concentration in 100% DMSO. This working stock of the compound was added to the assay well as the first component in the reaction, followed by the remaining components as detailed in the assay protocol listed above. The reaction was initiated by the addition of the MgATP mix. The kinase reaction was performed at room temperature for 40 minutes in presence of 250 μM substrate, 10 mM MgAcetate, [γ-33P-ATP] (specific activity approx. 500 cpm/pmol, concentration as required) and variable test article concentrations. The ATP concentrations in the assays were with 15 μM of the apparent. The reaction was stopped by the addition of 3% phosphoric acid solution. 10 μL of the reaction is then spotted onto a P30 filtermat and washed three times for 5 minutes in 75 mM phosphoric acid and once in methanol prior to drying and scintillation counting. In addition positive control wells contain all components of the reaction, except the compound of interest; however, DMSO (at a final concentration of 2%) were included in these wells to control for solvent effects as well as blank wells contain all components of the reaction, with a reference inhibitor replacing the compound of interest. This abolishes kinase activity and establishes the base-line (0% kinase activity remaining). The potency of each compound was reported by estimating the EC₅₀.

TABLE 2b
Inhibition of Btk

| Compound | EC₅₀ (nM) |
|---|---|
| 19 | a |
| 20 | a |
| 21 | a |
| 22 | a |
| 24 | b |
| 25 | a |
| 26 | a |

a - EC₅₀ <100 nM;
b - 100 nM < EC₅₀ < 1000 nM,
c - EC₅₀ >1000 nM.

Cellular Assay

Splenic Cell Proliferation Assay

Proliferation of splenocytes in response to anti-IgM can be blocked by inhibition of Btk. Splenocytes were obtained from 6 week old male CD1 mice (Charles River Laboratories Inc.). Mouse spleens were manually disrupted in PBS and filtered using a 70 um cell strainer followed by ammonium chloride red blood cell lysis. Cells were washed, resuspended in Splenocyte Medium (HyClone RPMI supplemented with 10% heat-inactivated FBS, 0.5× non-essential amino acids, 10 mM HEPES, 50 uM beta mercaptoethanol) and incubated at 37° C., 5% CO₂ for 2 h to remove adherent cells. Suspension cells were seeded in 96 well plates at 50,000 cells per well and incubated at 37° C., 5% CO₂ for 1 h. Splenocytes were pre-treated in triplicate with 10,000 nM curves of Formula I compounds for 1 h, followed by stimulation of cell proliferation with 2.5 ug/ml anti-IgM F(ab′)₂ (Jackson ImmunoResearch) for 72 h. Cell proliferation was measured by Cell Titer-Glo Luminescent Assay (Promega). EC₅₀ values (50% proliferation in the presence of compound as compared to vehicle treated controls) were calculated from dose response compound curves using GraphPad Prism Software.

EC₅₀ values are reported in Table 3:

TABLE 3
Inhibition of splenic cell proliferation

| Compound | EC₅₀ (nM) |
|---|---|
| 1 | a |
| 2 | a |
| 3 | a |
| 4 | a |
| 5 | a |
| 6 | a |
| 7 | b |
| 8 | b |
| 9 | b |
| 10 | b |
| 11 | b |
| 12 | b |
| 13 | b |
| 14 | b |
| 15 | b |
| 17 | a |
| 18 | a |
| 19 | a |
| 20 | b |
| 21 | a |
| 22 | b |
| 23 | a |
| 24 | a |
| 25 | a |
| 26 | a |

a - EC₅₀ <100 nM;
b - 100 nM < EC₅₀ < 1000 nM,
c - EC₅₀ >1000 nM,

1. A compound of Formula I: 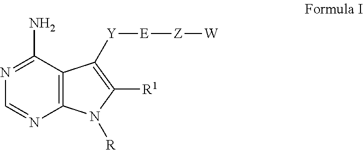
or pharmaceutically acceptable salt, solvate, solvate of salt, stereoisomer, tautomer, isotope, prodrug, complexe or biologically active metabolite thereof, wherein
R is selected from the group consisting of:
1) hydrogen,
2) alkyl,
3) heteroalkyl,
4) carbocyclyl,
5) heterocyclyl
6) aryl, or
7) heteroaryl;
wherein the alkyl, heteroalkyl, carbocyclyl, heterocycyl, aryl, or heteroaryl are optionally substituted;
R¹ is selected from the group consisting of:
1) hydrogen,
2) alkyl,
3) heteroalkyl,
4) carbocyclyl,
5) heterocyclyl, or
6) halogen,
wherein the alkyl, heteroalkyl, carbocyclyl, or heterocyclyl are optionally substituted;
Y is 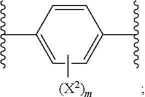
E is oxygen;
Z is 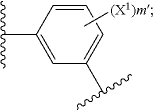
W is selected from
1) —OCH₂R², or
2) —CH₂OR²;
wherein R² is substituted or unsubstituted aryl, substituted or unsubstituted heteroaryl;
wherein Y-E-Z—W is 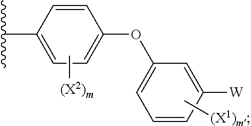
X¹ and X² are independently hydrogen or halogen;
m is an integer from 0 to 4,
m′ is an integer from 0 to 4.

2. The compound according to claim 1, wherein R is selected from the group consisting of:

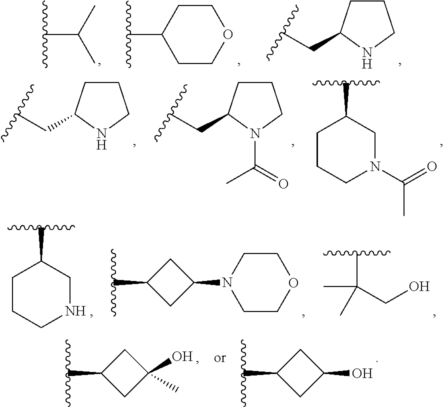

3. The compound according to claim 1, wherein R¹ is hydrogen.

4. The compound according to claim 1, wherein Z is selected from the group consisting of:

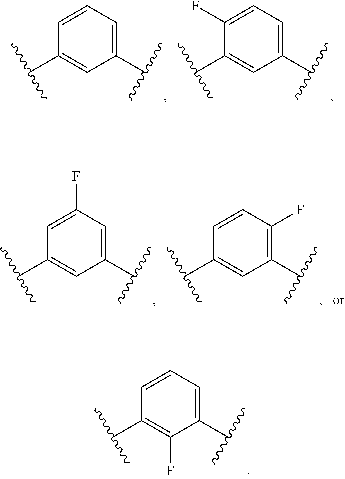 [SMILES: *c1cccc(*)c1 ; *c1ccc(F)c(*)c1 ; *c1cc(*)cc(F)c1 ; *c1ccc(F)c(*)c1 ; *c1cccc(*)c1F]

5. The compound according to claim 1, wherein Y is

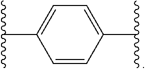 [SMILES: *c1ccc(*)cc1]

6. The compound according to claim 1, wherein W is selected from the group consisting of:

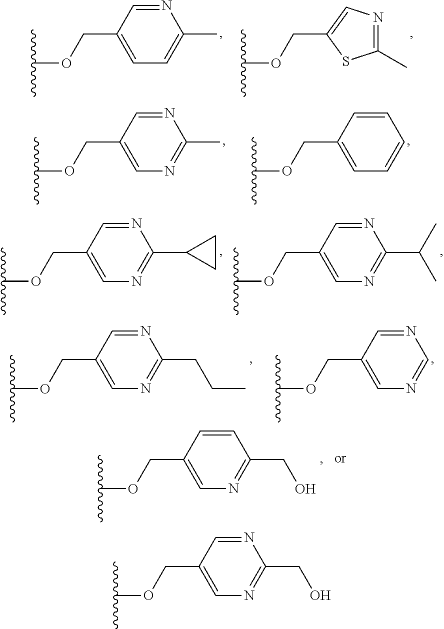

7. A compound selected from the group consisting of:

| Compound | Structure |
|---|---|
| 1 | 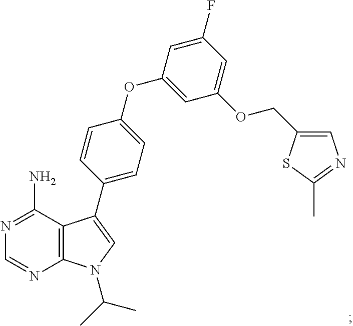 [SMILES: Cc1ncc(COc2cc(F)cc(Oc3ccc(-c4cn(C(C)C)c5ncnc(N)c45)cc3)c2)s1] |
| 2 | 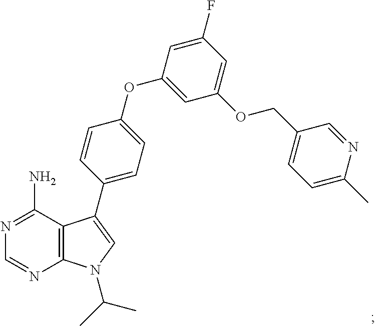 [SMILES: Cc1ccc(COc2cc(F)cc(Oc3ccc(-c4cn(C(C)C)c5ncnc(N)c45)cc3)c2)cn1] |
| 3 | 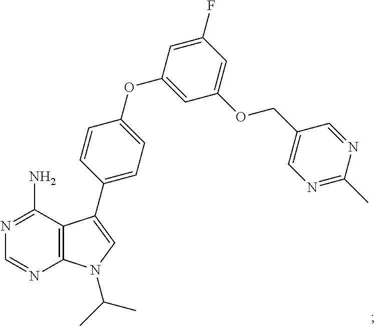 [SMILES: Cc1ncc(COc2cc(F)cc(Oc3ccc(-c4cn(C(C)C)c5ncnc(N)c45)cc3)c2)cn1] |
| 4 | 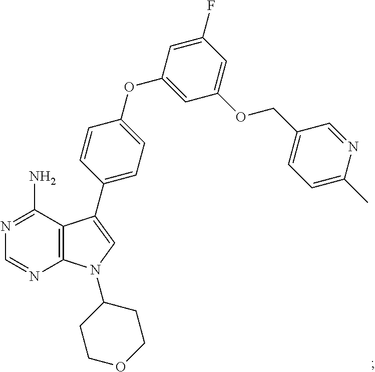 [SMILES: Cc1ccc(COc2cc(F)cc(Oc3ccc(-c4cn(C5CCOCC5)c5ncnc(N)c45)cc3)c2)cn1] |
| 5 | 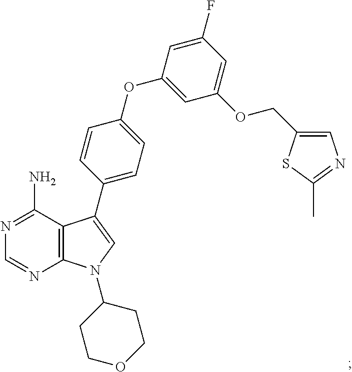 [SMILES: Cc1ncc(COc2cc(F)cc(Oc3ccc(-c4cn(C5CCOCC5)c5ncnc(N)c45)cc3)c2)s1] |
| 6 | 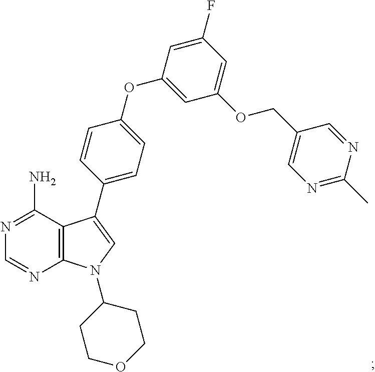 [SMILES: Cc1ncc(COc2cc(F)cc(Oc3ccc(-c4cn(C5CCOCC5)c5ncnc(N)c45)cc3)c2)cn1] |
| 7 | 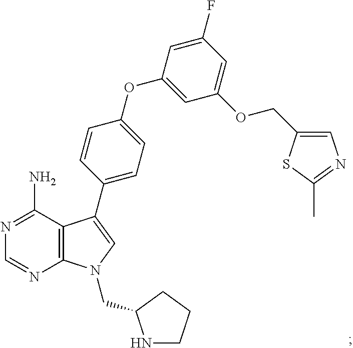 [SMILES: Cc1ncc(COc2cc(F)cc(Oc3ccc(-c4cn(C[C@@H]5CCCN5)c5ncnc(N)c45)cc3)c2)s1] |
| 8 | 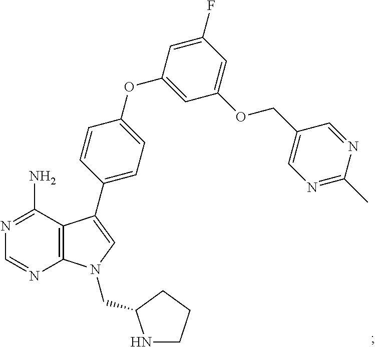 [SMILES: Cc1ncc(COc2cc(F)cc(Oc3ccc(-c4cn(C[C@@H]5CCCN5)c5ncnc(N)c45)cc3)c2)cn1] |
| 9 | 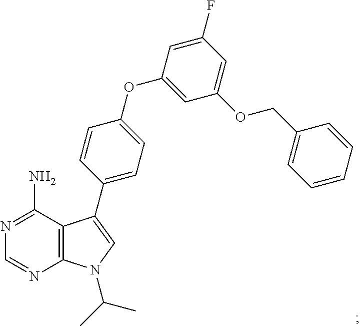 [SMILES: CC(C)n1cc(-c2ccc(Oc3cc(F)cc(OCc4ccccc4)c3)cc2)c2c(N)ncnc21] |
| 10 | 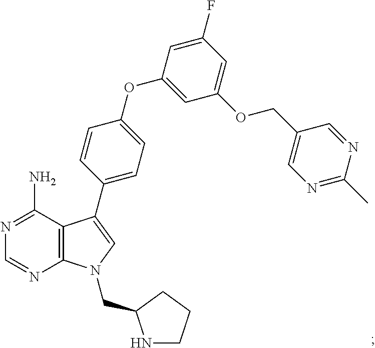 [SMILES: Cc1ncc(COc2cc(F)cc(Oc3ccc(-c4cn(C[C@H]5CCCN5)c5ncnc(N)c45)cc3)c2)cn1] |
| 11 | 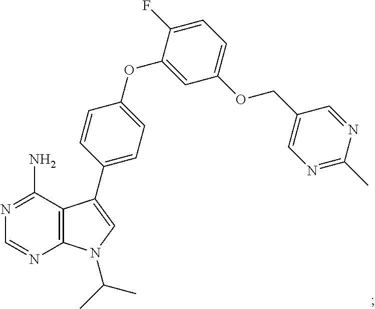 [SMILES: Cc1ncc(COc2ccc(F)c(Oc3ccc(-c4cn(C(C)C)c5ncnc(N)c45)cc3)c2)cn1] |
| 12 | 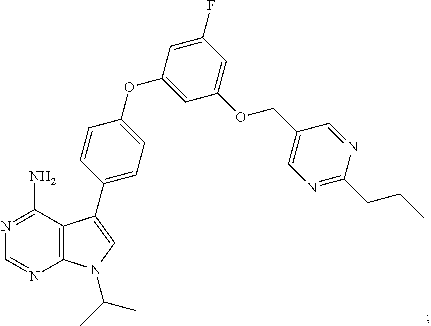 [SMILES: CCCc1ncc(COc2cc(F)cc(Oc3ccc(-c4cn(C(C)C)c5ncnc(N)c45)cc3)c2)cn1] |
| 13 | 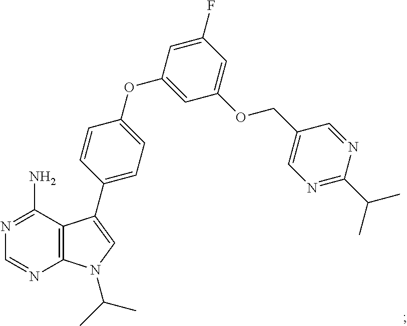 [SMILES: CC(C)c1ncc(COc2cc(F)cc(Oc3ccc(-c4cn(C(C)C)c5ncnc(N)c45)cc3)c2)cn1] |
| 14 | 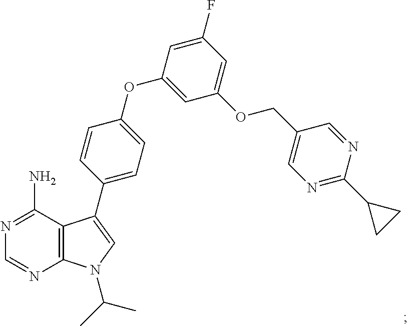 [SMILES: CC(C)n1cc(-c2ccc(Oc3cc(F)cc(OCc4cnc(C5CC5)nc4)c3)cc2)c2c(N)ncnc21] |
| 15 | 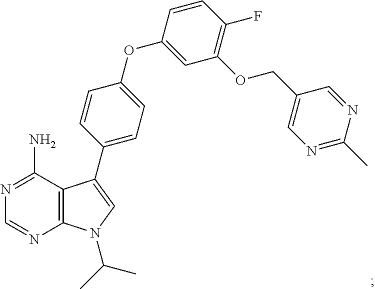 [SMILES: Cc1ncc(COc2cc(Oc3ccc(-c4cn(C(C)C)c5ncnc(N)c45)cc3)ccc2F)cn1] |
| 16 | 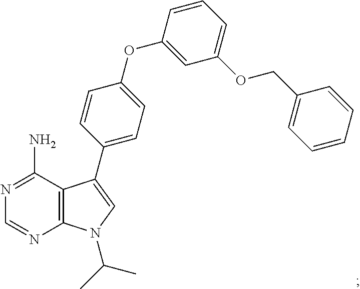 [SMILES: CC(C)n1cc(-c2ccc(Oc3cccc(OCc4ccccc4)c3)cc2)c2c(N)ncnc21] |
| 17 | 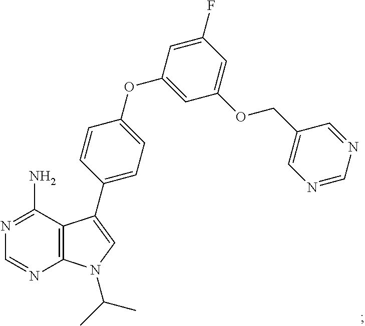 [SMILES: CC(C)n1cc(-c2ccc(Oc3cc(F)cc(OCc4cncnc4)c3)cc2)c2c(N)ncnc21] |
| 18 | 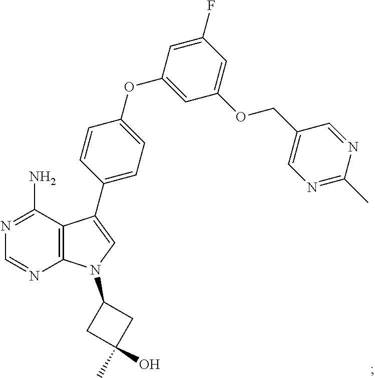 [SMILES: Cc1ncc(COc2cc(F)cc(Oc3ccc(-c4cn([C@H]5C[C@@](C)(O)C5)c5ncnc(N)c45)cc3)c2)cn1] |
| 19 | 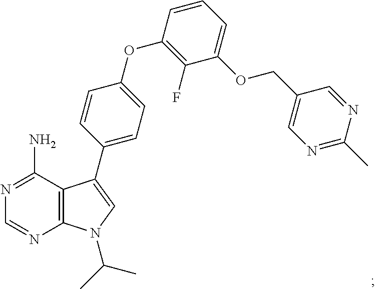 [SMILES: Cc1ncc(COc2cccc(Oc3ccc(-c4cn(C(C)C)c5ncnc(N)c45)cc3)c2F)cn1] |
| 20 | 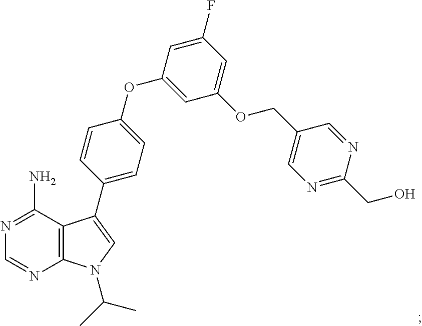 [SMILES: CC(C)n1cc(-c2ccc(Oc3cc(F)cc(OCc4cnc(CO)nc4)c3)cc2)c2c(N)ncnc21] |
| 21 | 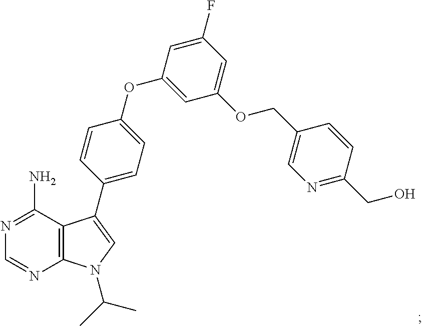 [SMILES: CC(C)n1cc(-c2ccc(Oc3cc(F)cc(OCc4ccc(CO)nc4)c3)cc2)c2c(N)ncnc21] |
| 22 | 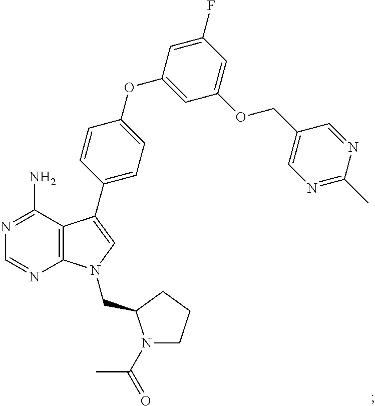 [SMILES: CC(=O)N1CCC[C@@H]1Cn1cc(-c2ccc(Oc3cc(F)cc(OCc4cnc(C)nc4)c3)cc2)c2c(N)ncnc21] |
| 23 | 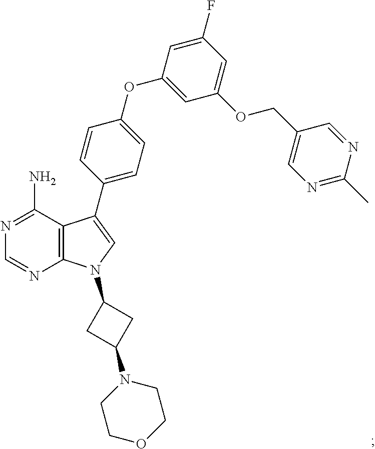 [SMILES: Cc1ncc(COc2cc(F)cc(Oc3ccc(-c4cn([C@H]5C[C@@H](N6CCOCC6)C5)c5ncnc(N)c45)cc3)c2)cn1] |
| 24 | 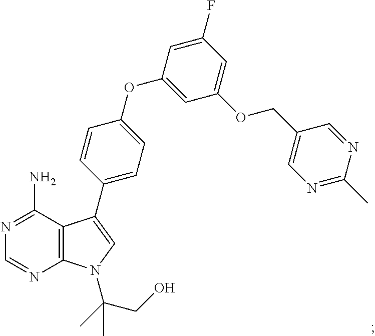 [SMILES: Cc1ncc(COc2cc(F)cc(Oc3ccc(-c4cn(C(C)(C)CO)c5ncnc(N)c45)cc3)c2)cn1] |
| 25 | 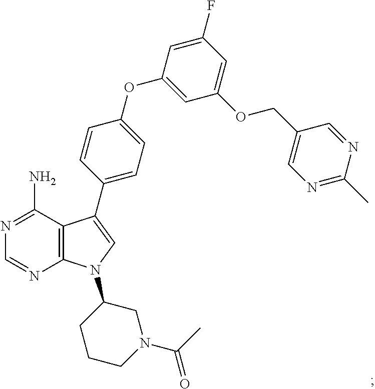 [SMILES: CC(=O)N1CCC[C@@H](n2cc(-c3ccc(Oc4cc(F)cc(OCc5cnc(C)nc5)c4)cc3)c3c(N)ncnc32)C1] |
| 26 | 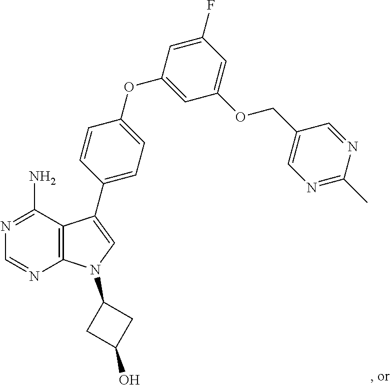 [SMILES: Cc1ncc(COc2cc(F)cc(Oc3ccc(-c4cn([C@H]5C[C@@H](O)C5)c5ncnc(N)c45)cc3)c2)cn1] |
| 27 | 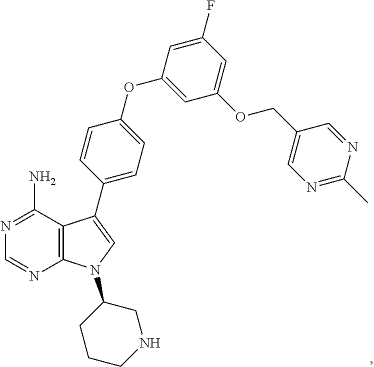 [SMILES: Cc1ncc(COc2cc(F)cc(Oc3ccc(-c4cn([C@@H]5CCCNC5)c5ncnc(N)c45)cc3)c2)cn1] |

or pharmaceutically acceptable salt, solvate, or solvate of salt thereof.

8. A process for preparing a compound of Formula I, wherein the process comprises:

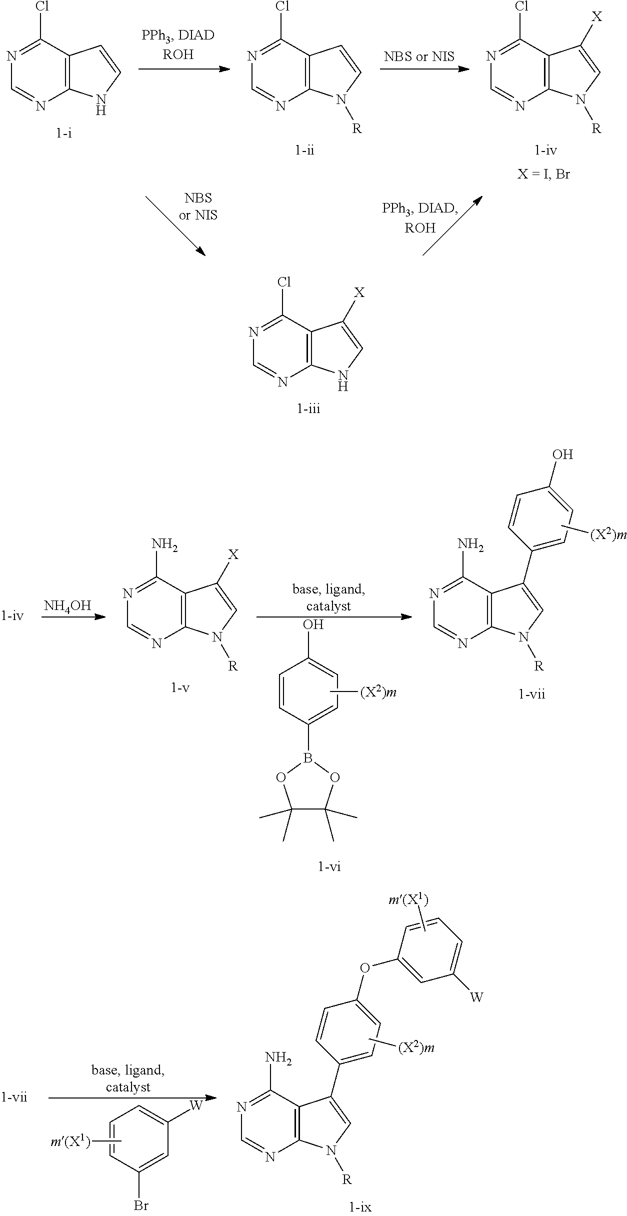

9. A process for preparing a compound of Formula I, wherein the process comprises:

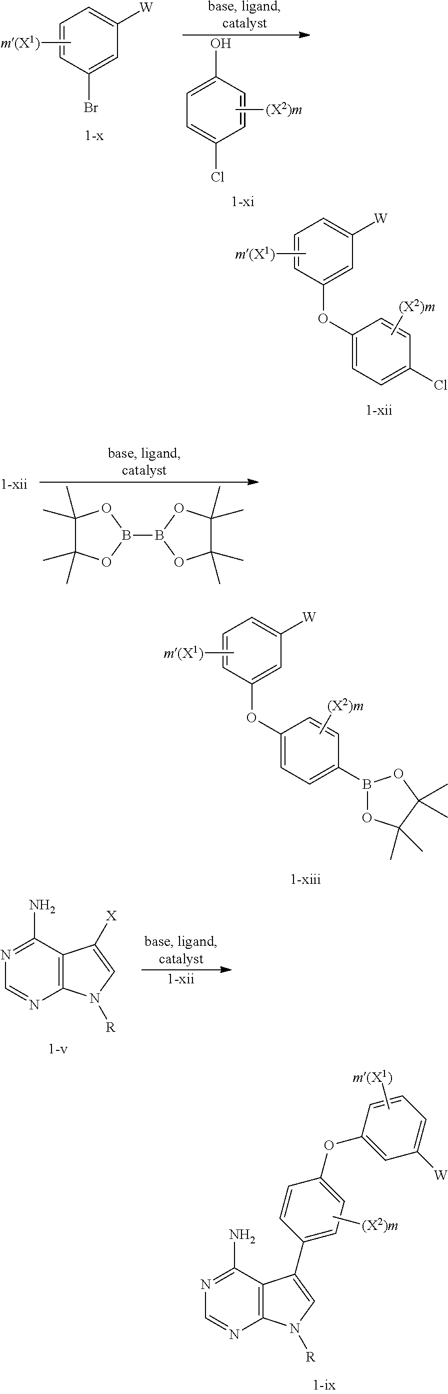

10. A method of treating a disease or disorder in a subject in need of such treatment, the method comprising administering to said subject azo compound of claim 1, or a pharmaceutically acceptable salt, or solvate thereof.

11. A method of treating proliferative, inflammatory, infectious, or autoimmune diseases in a subject in need of such treatment, the method comprising administering to said subject a compound of claim 1, or a pharmaceutically acceptable salt, or solvate thereof.

12. The method of claim 11, wherein the proliferative disease is cancer.

13. The method of claim 11, wherein said compound is an inhibitor of a protein kinase.

14. A method of treating a subject suffering from a protein kinase mediated disease, disorder, or condition in which Tec kinase family member activity is implicated, the method comprising administering to said subject a therapeutically effective amount of a compound of claim 1, or a pharmaceutically acceptable salt or solvate thereof.

15. A method of treating a subject suffering from a protein kinase mediated disease, disorder, or condition in which Src kinase family member activity is implicated, the method comprising administering to said subject a therapeutically effective amount of a compound of claim 1, or a pharmaceutically acceptable salt or solvate thereof.

16. A method of treating a subject suffering from a protein kinase mediated disease, disorder, or condition is associated with inhibiting a Btk kinase activity, the method comprising administering to said subject a therapeutically effective amount of a compound of claim 1, or a pharmaceutically acceptable salt or solvate thereof.

17. A method of treating a proliferative, inflammatory, autoimmune, or infectious diseases in a subject in need of such treatment, the method comprising administering to said subject a therapeutically effective amount of a compound of claim 1, or a pharmaceutically acceptable salt or solvate thereof.

18. A method of treating a proliferative disorder, or disease state, in a subject in need of such treatment, the method comprising administering to said subject a therapeutically effective amount of a compound of claim 1, or a pharmaceutically acceptable salt or solvate thereof in combination with an agent selected from: an estrogen receptor modulator; an androgen receptor modulator; a retinoid receptor modulator; a cytotoxic agent; an anti-proliferative agent comprises adriamycin, dexamethasone, vincristine, cyclophosphamide, fluorouracil, topotecan, taxol, interferons, or platinum derivatives; an anti-inflammatory agent comprises corticosteroids, TNF blockers, IL-1 RA, azathioprine, cyclophosphamide, or sulfasalazine; a prenyl-protein transferase inhibitor; an HMG-CoA reductase inhibitor; an HIV protease inhibitor; a reverse transcriptase inhibitor; an angiogenesis inhibitor comprises sorafenib, sunitinib, pazopanib, or everolimus; an immunomodulatory, or immunosuppressive agents comprises cyclosporin, tacrolimus, rapamycin, mycophenolate mofetil, interferons, corticosteroids, cyclophophamide, azathioprine, or sulfasalazine; a PPAR-γ agonist comprising thiazolidinediones; a PPAR-δ agonist; an inhibitor of inherent multidrug resistance; an agent for the treatment of anemia, comprising erythropoiesis-stimulating agents, vitamins, or iron supplements; an anti-emetic agent including 5-HT3 receptor antagonists, dopamine antagonists, NK1 receptor antagonist, H1 histamine receptor antagonists, cannabinoids, benzodiazepines, anticholinergic agents, or steroids; an agent for the treatment of neutropenia; an immunologic-enhancing agents; a proteasome inhibitors; an HDAC inhibitors; an inhibitor of the chemotrypsin-like activity in the proteasome; a E3 ligase inhibitors; a modulator of the immune system including interferon-alpha, Bacillus Calmette-Guerin (BCG), or ionizing radition (UVB) that can induce the release of cytokines, interleukins, TNF, or induce release of death receptor ligands including TRAIL; a modulator of death receptors TRAIL, or TRAIL agonists including humanized antibodies HGS-ETR1, or HGS-ETR2; neurotrophic factors selected from cetylcholinesterase inhibitors, MAO inhibitors, interferons, anti-convulsants, ion channel blockers, or riluzole; Anti-Parkinsonian agents comprising anticholinergic agents, or dopaminergic agents, including dopaminergic precursors, monoamine oxidase B inhibitors, COMT inhibitors, dopamine receptor agonists; agents for treating cardiovascular disease comprises beta-blockers, ACE inhibitors, diuretics, nitrates, calcium channel blockers, or statins; agents for treating liver disease comprises corticosteroids, cholestyramine, or interferons; anti-viral agents, including nucleoside reverse transcriptase inhibitors, non-nucleoside reverse transcriptase inhibitors, protease inhibitors, integrase inhibitors, fusion inhibitors, chemokine receptor antagonists, polymerase inhibitors, viral proteins synthesis inhibitors, viral protein modification inhibitors, neuraminidase inhibitors, fusion or entry inhibitors; agents for treating blood disorders comprising corticosteroids, anti-leukemic agents, or growth factors; agents for treating immunodeficiency disorders comprising gamma globulin, adalimumab, etarnecept, or infliximab; a HMG-CoA reductase inhibitors including torvastatin, fluvastatin, lovastatin, pravastatin, rosuvastatin, simvastatin, or pitavastatin, or in combination, or sequentially with radiation, or at least one chemotherapeutic agent.

19. A method of treating a proliferative disorder, or disease state in a subject in need of such treatment, the method comprising administering to said subject a therapeutically effective amount of a compound of claim 1, or a pharmaceutically acceptable salt or solvate thereof in combination with a death receptor agonist.

20. A method of treating or preventing arthritis, or immune hypersensitivity in a subject in need of such treatment, the method comprising administering to said subject a therapeutically effective amount of a compound of claim 1, or a pharmaceutically acceptable salt or solvate thereof.

21. A method of treating or preventing an autoimmune disease in a subject in need of such treatment, the method comprising administering to said subject a therapeutically effective amount of a compound of claim 1, or a pharmaceutically acceptable salt or solvate thereof.

22. A method of treating or preventing infectious diseases, or inflammation in a subject in need of such treatment, the method comprising administering to said subject a therapeutically effective amount of a compound of claim 1, or a pharmaceutically acceptable salt or solvate thereof.

23. A method of treating or preventing heart attacks, or stroke in a subject in need of such treatment, the method comprising administering to said subject a therapeutically effective amount of a compound of claim 1, or a pharmaceutically acceptable salt or solvate thereof.

24. A pharmaceutical composition comprising a compound of claim 1, or a pharmaceutically acceptable salt, solvate, solvate of salt, stereoisomer, tautomer, isotope, prodrug, complex or biologically active metabolite thereof, and at least one pharmaceutically acceptable carrier, diluents, or excipient.

25. A method of treating a subject suffering from a protein kinase mediated disease, disorder, or condition in which Tyrosine kinase family member activity is implicated, the method comprising administering to said subject the pharmaceutical composition of claim 24.

26. A method of treating a subject suffering from a protein kinase mediated disease, disorder, or condition associated with Src kinase family members, the method comprising administering to said subject the pharmaceutical composition of claim 24.

27. A method of a subject suffering from a protein kinase mediated disease, disorder, or condition, wherein a protein kinase mediated disease, is associated with inhibiting a Btk kinase activity, the method comprising administering to said subject the pharmaceutical composition of claim 24.

28. A method of treating a subject suffering from a protein kinase mediated disease, disorder, or condition in which Tyrosine kinase family member activity is implicated, the method comprising administering to said subject the pharmaceutical composition of claim 24 alone or in combination with at least one other pharmaceutically acceptable agent.

29. A method of treating or preventing a proliferative, inflammatory or autoimmune disease including: cancer; psoriasis or dermatological disorders; viral disorders; cardiovascular diseases: restenosis or cardiomyopathy; CNS disorders; glomerulonephritis or rheumatoid arthritis; hormone-related diseases; metabolic disorders; stroke; alopecia, emphysema; inflammatory diseases; infectious or fungal diseases, malaria or parasitic disorders, in a subject in need of such treatment the method comprising administering to said subject the pharmaceutical composition of claim 24.

30. A method of modulating kinase activity in a human or animal subject, the method comprising administering to said subject the pharmaceutical composition of claim 24.

31. A method of inhibiting protein kinase activity in a human or animal cell, or tissue, the method comprising administering to said human or animal cell, or tissue the compound of claim 1.

32. (canceled)

33. A probe comprising a compound of claim 1, or a detectable label, or affinity tag for said compound.

34. The probe according to claim 33, wherein the detectable label is selected from the group consisting of: a fluorescent moiety, a chemiluminescent moiety, a paramagnetic contrast agent, a metal chelate, a radioactive isotope-containing moiety, or biotin.
